(19) United States
(12) Patent Application Publication
(10) Pub. No.: US 2024/0010734 A1
(43) Pub. Date: Jan. 11, 2024

(54) UNGULATE-DERIVED POLYCLONAL IMMUNOGLOBULIN SPECIFIC FOR EGFR AND USES THEREOF

(71) Applicant: SAB, LLC, Sioux Falls, SD (US)

(72) Inventors: Hua WU, Sioux Falls, SD (US); Eddie J. SULLIVAN, Sioux Falls, SD (US); Kristi A. Egland, Sioux Falls, SD (US); Christoph L. BAUSCH, Sioux Falls, SD (US)

(73) Assignee: SAB, LLC, Sioux Falls, SD (US)

(21) Appl. No.: 18/036,591

(22) PCT Filed: Nov. 12, 2021
(86) PCT No.: PCT/US2021/059225

§ 371 Date: May 11, 2023

Related U.S. Application Data
(60) Provisional application No. 63/113,643, filed on Nov. 13, 2020.

(51) Int. Cl. C07K 16/28; C07K 16/30; A61P 35/00

(52) U.S. Cl. CPC C07K 16/2863; C07K 16/3023; A61P 35/00; C07K 2317/20; C07K 2317/10; C07K 2317/732; C07K 2317/734; C07K 2317/76

(57) ABSTRACT

Provided are human polyclonal immunoglobulin products specific for Epidermal Growth Factor Receptor (EGFR) for use in treating or preventing cancer. Further provided are methods for making such compositions in a transgenic ungulate, e.g. using a transchromosomic bovine (TcB) system.

### CROSS-REFERENCE TO RELATED APPLICATIONS

This application claims priority to U.S. application Ser. No. 63/113,635, filed Nov. 13, 2020, which is hereby incorporated by reference in its entirety herein.

### INCORPORATION BY REFERENCE OF SEQUENCE LISTING

This application contains a Sequence Listing which has been submitted in ASCII format via EFS-WEB and is hereby incorporated by reference in its entirety. Said ASCII copy, created on Nov. 13, 2021, is named SABB_004_01WO_ST25.txt and is 83 kilobytes in size.

### TECHNICAL FIELD

The invention relates to polyclonal immunoglobulin products for treatment of cancer.

### BACKGROUND

For over half a century, physicians have relied on surgery, chemotherapy, and radiotherapy as the main weapons to fight cancer. Recently, new immunotherapies have been added to the arsenal and are quickly becoming routine therapy for cancer patients.

Epidermal Growth Factor Receptor (EGFR) is a surface-expressed protein receptor involved in proliferation of both normal and cancer cells. It is found in high levels in some cancers. Several anti-EGFR monoclonal antibodies (cetuximab, panitumumab, nimotuzumab, and necitumumab) are approved for treatment of EGFR+cancer.

There exists a need for immunoglobulin products for therapeutic use in patients suffering from or at risk for diseases and disorders including but not limited to cancer.

### SUMMARY

The present inventors have developed a polyclonal human immunoglobulin product for treatment of disease associated with the Epidermal Growth Factor Receptor (EGFR) made in ungulates (e.g., bovines) genetically engineered to produce polyclonal human immunoglobulin having a human polypeptide sequence, representative samples of which were deposited under the Budapest treaty on Nov. 2, 2021 with the American Type Culture Collection (ATCC) and given the Deposit No. PTA-127158. An anti-EGFR human polyclonal product may have substantial therapeutic and safety benefits compared to monoclonal antibody therapy.

In one aspect, the disclosure provides an ungulate-derived polyclonal human immunoglobulin composition, comprising a population of human immunoglobulins, wherein the population of human immunoglobulins specifically binds Epidermal Growth Factor Receptor (EGFR).

In some embodiments, the composition is produced by immunizing a transgenic ungulate with an antigenic fragment of EGFR.

In some embodiments, the antigenic fragment of EGFR is an EGFR extracellular domain.

In some embodiments, the antigenic fragment comprises, consists of, or consists essentially of an EGFR sequence according to SEQ ID NO: 16, amino acids 25-638 of an EGFR sequence according to SEQ ID NO: 15, or variants thereof.

In some embodiments, the antigenic fragment shares at least 80%, 90%, 95%, 96%, 97%, 98%, 99%, or 100% identity to SEQ ID NO: 16, SEQ ID NO: 15, or fragments thereof.

In some embodiments, the population of human immunoglobulins binds to a non-small cell lung cancer cell, optionally one or more of MDA-MB-468, H292, H460, H1975, HCC827, and H1299 cells.

In some embodiments, the population of human immunoglobulins exhibit complement-dependent-cytotoxicity (CDC) activity.

In some embodiments, the population of human immunoglobulins exhibit antibody-dependent cellular toxicity (ADCC) activity.

In some embodiments, the population of human immunoglobulins block EGF ligand from binding to the EGF receptor.

In some embodiments, the population of human immunoglobulins blocks the EGF receptor signaling pathway.

In some embodiments, the population of human immunoglobulin decreases the phosphorylation status of Erk1/2.

In some embodiments, the population of human immunoglobulin decreases the phosphorylation status of Akt.

In some embodiments, the population of human immunoglobulins inhibits tumor cell growth in vivo.

In some embodiments, the population of human immunoglobulins has an avidity for EGFR of at least 0.1 1/sec, at least 0.01 1/sec, at least 0.001 1/sec at least 0.0001 1/sec, or at least 0.00001 1/sec.

In some embodiments, the population of human immunoglobulins has an avidity for EGFR of 0.1 to 0.01 1/sec, 0.01 to 0.001 1/sec, 0.001 to 0.0001 1/sec, or 0.0001 to 0.00001 1/sec.

In some embodiments, the population of human immunoglobulin composition is substantially similar to ATCC Deposit No. PTA-127158 or wherein population of human immunoglobulins has an avidity for EGFR at least 50%, at least 60%, at least 70%, at least 80%, at least 90%, at least 100%, at least 110%, or at least 120% as great as that of ATCC Deposit No. PTA-127158.

In another aspect, the disclosure provides a method of making polyclonal human immunoglobulin specific for Epidermal Growth Factor Receptor (EGFR), comprising administering an antigenic fragment of EGFR, or a polynucleotide encoding the antigenic fragment, to a transgenic ungulate, wherein the transgenic ungulate comprises a genome comprising a human immunoglobulin locus or an artificial chromosome comprising a human immunoglobulin locus, and wherein the transgenic ungulate produces a population of human immunoglobulins that specifically binds EGFR.

In some embodiments, the method comprises administering the antigenic fragment or polynucleotide encoding the antigenic fragment 3, 4, 5, or more times.

In some embodiments, the method comprises collecting serum or plasma from the transgenic ungulate.

In some embodiments, the serum or plasma comprises a population of fully human immunoglobulins.

In some embodiments, the antigenic fragment of EGFR is an EGFR extracellular domain.

In some embodiments, the antigenic fragment comprises, consists of, or consists essentially of an EGFR sequence according to SEQ ID NO: 16, amino acids 25-638 of an EGFR sequence according to SEQ ID NO: 15, or variants thereof.

In some embodiments, the antigenic fragment shares at least 80%, 90%, 95%, 96%, 97%, 98%, 99%, or 100% identity to SEQ ID NO: 16, SEQ ID NO: 15, or fragments thereof.

In some embodiments, the population of human immunoglobulins binds a non-small cell lung cancer cell, optionally one or more of MDA-MB-468, H292, H460, H1975, HCC827, and H1299 cells.

In some embodiments, the population of human immunoglobulins exhibit complement-dependent-cytotoxicity (CDC) activity.

In some embodiments, the population of human immunoglobulins exhibit antibody-dependent cellular toxicity (ADCC) activity.

In some embodiments, the population of human immunoglobulins block EGF ligand from binding to the EGF receptor.

In some embodiments, the population of human immunoglobulins blocks the EGF receptor signaling pathway.

In some embodiments, the population of human immunoglobulin decreases the phosphorylation status of Erk1/2.

In some embodiments, the population of human immunoglobulin decreases the phosphorylation status of Akt.

In some embodiments, the population of human immunoglobulins inhibits tumor cell growth in vivo.

In some embodiments, the population of human immunoglobulins has an avidity for EGFR of at least 0.1 1/sec, at least 0.01 1/sec, at least 0.001 1/sec at least 0.0001 1/sec, or at least 0.00001 1/sec.

In some embodiments, the population of human immunoglobulins has an avidity for EGFR of 0.1 to 0.01 1/sec, 0.01 to 0.001 1/sec, 0.001 to 0.0001 1/sec, or 0.0001 to 0.00001 1/sec.

In some embodiments, the population of human immunoglobulin composition is substantially similar to ATCC Deposit No. PTA-127158 or wherein population of human immunoglobulins has an avidity for EGFR at least 50%, at least 60%, at least 70%, at least 80%, at least 90%, at least 100%, at least 110%, or at least 120% as great as that of ATCC Deposit No. PTA-127158.

In some embodiments, the antigenic fragment is administering in a pharmaceutical composition comprising Montanide ISA-206 and/or Quil A.

In some embodiments, the method comprises a) administering a polynucleotide encoding the antigenic fragment of EGFR; b) administering a polynucleotide encoding the encoding the antigenic fragment of EGFR, three to four weeks later; c) administering the antigenic fragment of EGFR, four weeks later d) administering the antigenic fragment of EGFR, four weeks later; and e) administering the antigenic fragment of EGFR, four weeks later.

In some embodiments, the method comprises purifying the human immunoglobulin to produce a composition of the disclosure.

In another aspect, the disclosure provides a pharmaceutical composition, comprising a composition of the disclosure and optionally one or more pharmaceutically acceptable excipients.

In another aspect, the disclosure provides a method of treating or preventing cancer in a subject in need thereof, comprising administering an effective amount of a composition of the disclosure or a pharmaceutical composition of the disclosure to the subject.

Additional embodiments, features, and advantages of the invention will be apparent from the following detailed description and through practice of the invention.

### BRIEF DESCRIPTION OF THE DRAWINGS

FIGS. 1A-1H show construction of the isHAC and isKcHACΔ vectors.

FIG. 1A shows a flow of the isHAC and isKcHACΔ vector construction. The bovinizing vector, pCC1BAC-isHAC, is BAC-based (backbone is pCC1BAC vector), consisting of 10.5 kb and 2 kb of genomic DNA as a long and short arm, respectively, 9.7 kb of the bovine genomic DNA covering the bovine Iγ1-Sγ1 and its surrounding region to replace the human corresponding 6.8 kb of Iγ1-Sγ1 region, the chicken β-actin promoter-driven neo gene flanked by FRT sequence and DT-A gene. After the targeted bovinization, the neo cassette is removed by FLP introduction.

FIG. 1B shows detailed information on the targeting vector pCC1BAC-isHAC. The 2 kb of Afe I-Bam HI fragment and 10.5 kb of Apa I-Hpa I fragment for a short arm and long arm were obtained from clone h10 and clone h18/h20, respectively, derived from λ, phage genomic library constructed from CHO cells containing the κHAC by screening using a probe around the human Iγ1-Sγ1 region. The 9.7 kb fragment (5′ end through Bsu36 I) was obtained from clone b42 derived from the λ phage bovine genomic library.

FIG. 1C shows genotyping of the bovinized Iγ1-Sγ1 region. Five sets of PCR primers that amplify genomic PCR were implemented, as indicated. The iscont1-F1/R1 primer set is a positive PCR specific to the homologous recombination. The iscont1-F1×hIgG1-R10 is a negative PCR that is prohibited by the presence of the neo cassette. isHAC-Sw-dig-F5/R3 and isHAC-TM-dig-F3/R2 are for structural integrity check of their corresponding region, digested by Barn HI+Pvu II and Age I, Sma I or Pvu II, respectively. The primer set, bNeo 5′-R×bIgG1-5′-seq-R6, is used to confirm the presence of FRT sequence.

FIG. 1D shows genotyping after the FLP-FRT deletion of the neo cassette.

FIG. 1E shows extensive genomic PCR for genotyping of the isHAC vector. Location of each genomic PCR primer pair is depicted in relation to the isHAC vector structure.

FIG. 1F shows CGH analysis among three different CHO clones containing the isHAC vector. DNA from isC1-133 was used as a reference. There was no apparent structural difference of the isHAC among the three cell lines.

FIG. 1G shows extensive genomic PCR for genotyping of the isKcHACΔ vector. Location of each genomic PCR primer pair is depicted in relation to the isKcHACΔ vector structure.

FIG. 1H shows CGH analysis among three different CHO clones containing the isKcHACΔ vector. DNA from isKCDC15-8 was used as a reference. There was no apparent structural difference of the isKcHACΔ among the three cell lines.

FIG. 2 shows an SDS-PAGE analysis of EGFR-hFc. After expression of pEGFR-hFc in FreeStyle 293F cells, supernatant containing the protein was harvested and EGRF-hFc was purified. Two mg of protein was loaded onto an SDS-PAGE gel, along with a molecular weight marker. The gel was developed using Coomassie blue staining. The arrow indicates the EGFR-hFc band.

FIG. 3 shows quantification of anti-EGFR antibodies in serum of a Tc bovine. Vaccinations (“V”) V1 to V4 and days after each vaccination (“D”), D0 to D10, are indicated. An indirect ELISA demonstrates anti-EGFR specific titers (y-axis) with corresponding vaccination number and serum harvest day (x-axis).

FIG. 4 shows indirect ELISA analysis indicating the titer of purified SAB-162E compared to negative control (NC) human IgG purified from Tc bovine pre-immune plasma.

FIG. 5A-5B show that SAB-162E binds cellular EGFR protein.

FIG. 5A shows the binding of SAB-162E to the breast cancer cell line, MDA-MB-468, which has high EGFR levels on the cell surface. The human leukemia cell line, HL-60, was used as a negative control because the cells do not express EGFR. Binding levels of SAB-162E were compared to cetuximab and naïve TcB polyclonal antibody.

FIG. 5B shows flow cytometry analysis of SAB-162E binding to EGFR positive NSCLC cells lines (H292, H460, H1975, HCC827, and H1299). Raji and Ramos lymphoma cells are EGFR negative. Bars represent mean fluorescence intensity (MFI) of≥3,000 singlet live cells on the y-axis. Cell lines are indicated on the x-axis. Representative data from one biological replicate of duplicate experiments is shown.

FIG. 6A-6C show human NSCLC cellular binding titrations of SAB-162E. SAB-162E (closed circles) binding levels were compared to the saturating MFI level of cetuximab (dotted line). The maximum fluorescence intensity (MFI) level is on the y-axis. Each serial antibody dilution represents≥3,000 singlet live cells for each data point. Representative data from one biological replicate of duplicate experiments is shown.

FIG. 6A shows the titration of SAB-162E binding to the NSCLC cell line, H292.

FIG. 6B shows the titration of SAB-162E binding to the NSCLC cell line, H1975.

FIG. 6C shows the titration of SAB-162E binding to the NSCLC cell line, H1299.

FIG. 7A-7E show SAB-162E induces antibody-dependent cell-mediated cytotoxicity (ADCC) activation of engineered Jurkat Lucia NFAT CD16 cells. Activation of effector cells is depicted by luciferase activity in RLU on the y-axis verses increasing SAB-162E (closed circles) antibody concentration depicted on the x-axis. SAB naïve negative control (NC) IgG is shown only at the highest antibody concentration (open circle). Data points are means of technical triplicates. Error bars indicate SD. Representative data from one replicate of biological duplicate experiments are shown.

FIG. 7A shows ADCC activity against target NSCLC cell line, H1299, using a 6:1 effector cell to target cell ratio (E:T cell ratio) after 48 hours.

FIG. 7B shows ADCC activity against the target NSCLC cell line, H460 (6:1 E:T cell ratio) after 24 hours.

FIG. 7C shows ADCC activity against the EGFR expressing NSCLC cell line, H1975 (6:1 E:T cell ratio) after 24 hours.

FIG. 7D shows ADCC activity against target NSCLC cell line, H292 (6:1 E:T cell ratio) after 24 hours.

FIG. 7E shows ADCC activity against target NSCLC cell line, HCC827 (6:1 E:T cell ratio) after 24 hours.

FIG. 8 shows ADCC activity against the target human breast cancer cell line, MDA-MB-468, which expresses high levels of EGFR. Three different antibodies were tested: cetuximab, SAB-162E, and naïve TcB as a negative control. Effector cells, PBMCs, were added at an effector cell to target cell (E:T cell ratio) of 20:1.

FIG. 9 shows complement-dependent cytotoxicity (CDC) activity of SAB-162E.

FIG. 10A-10B show that SAB-162E blocks EGF binding to EGFR expressing human NSCLC cell lines. Cells were pre-incubated with increasing amounts of SAB-162E. Saturating levels of EGF-Alexa Fluor conjugate were added, and the mean fluorescence intensity (MFI) was measured by flow cytometry. SAB-162E blockage of EGF binding is indicated by closed circles. The negative control (NC) IgG is shown only at the highest concentration (open circle). Maximum binding is demonstrated with saturating EGF levels and no antibody blockage (open triangle). Data points are MFI from≥3,000 singlet live cells. Representative data from one experiment of biological duplicate experiments is shown for each NSCLC cell line.

FIG. 10A shows MFI of EGF-Alexa Fluor conjugate binding to NSCLC cells line, H1299, preincubated with increasing concentrations of SAB-162E.

FIG. 10B shows MFI of EGF-Alexa Fluor conjugate binding to NSCLC cells line, H292, preincubated with increasing concentrations of SAB-162E.

FIG. 11A-11G show that SAB-162E blockade of EGF binding inhibits EGFR downstream signaling. H292 NSCLC cells were incubated with increasing concentrations of SAB-162E and subsequently stimulated with EGF prior to collecting cellular lysates. Western immunoblot analysis of the phosphorylation status of the EGFR downstream pathway proteins, Erk and Akt, are represented as indicated.

FIG. 11A shows a western immunoblot analysis of cell lysates derived from EGF stimulated H292 cells incubated with SAB-162E. The membrane containing the transferred lysates was probed with antibodies against Erk1/2, phosphorylated Erk1/2, Akt and phosphorylated Akt.

FIG. 11B shows densitometry of Erk1/2.

FIG. 11C shows densitometry of phosphorylated Erk1/2.

FIG. 11D shows the relative phosphorylated Erk1/2 to total Erk1/2 from 11B and 11C.

FIG. 11E shows the densitometry of AKT.

FIG. 11F shows the densitometry of phosphorylated AKT.

FIG. 11G shows the relative phosphorylated AKT to total AKT from 11E and 11F

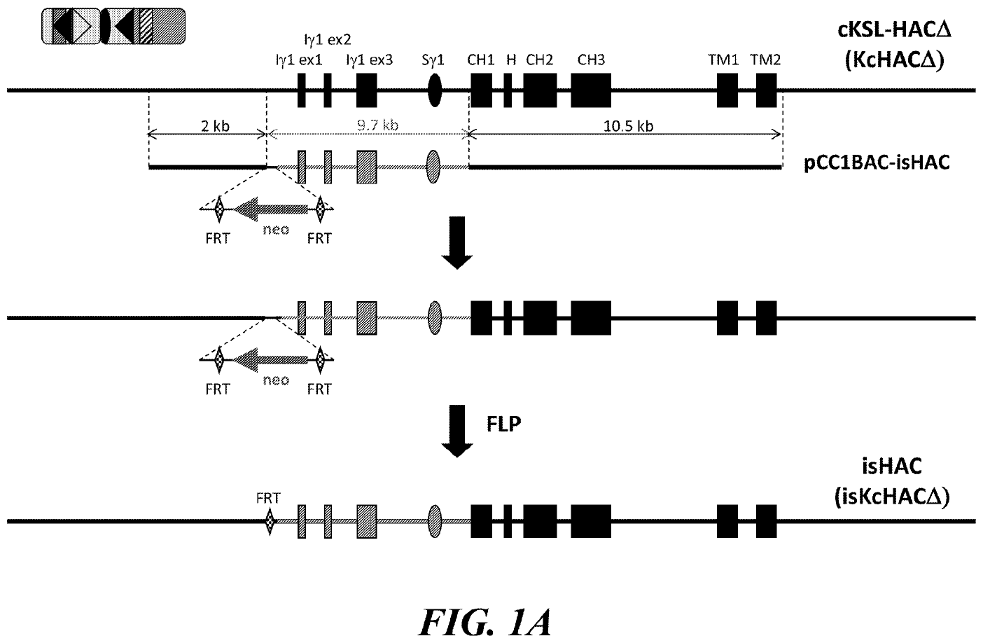

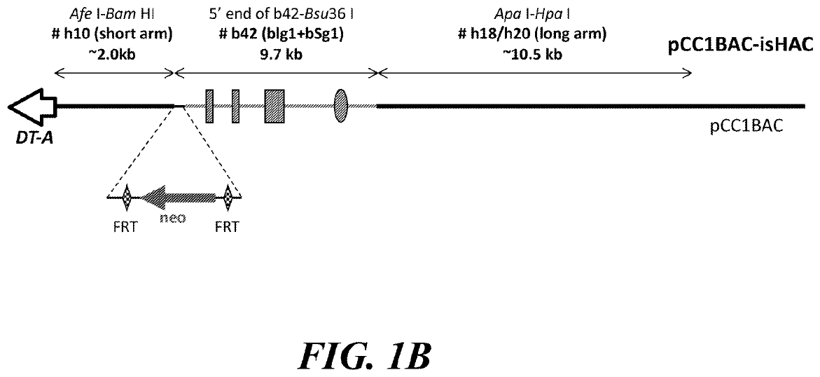

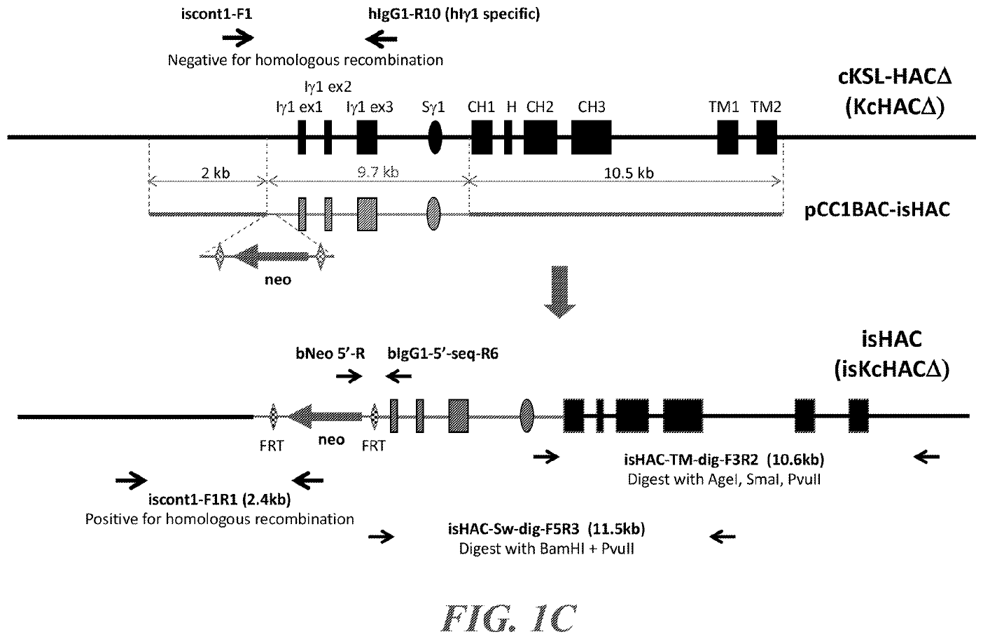

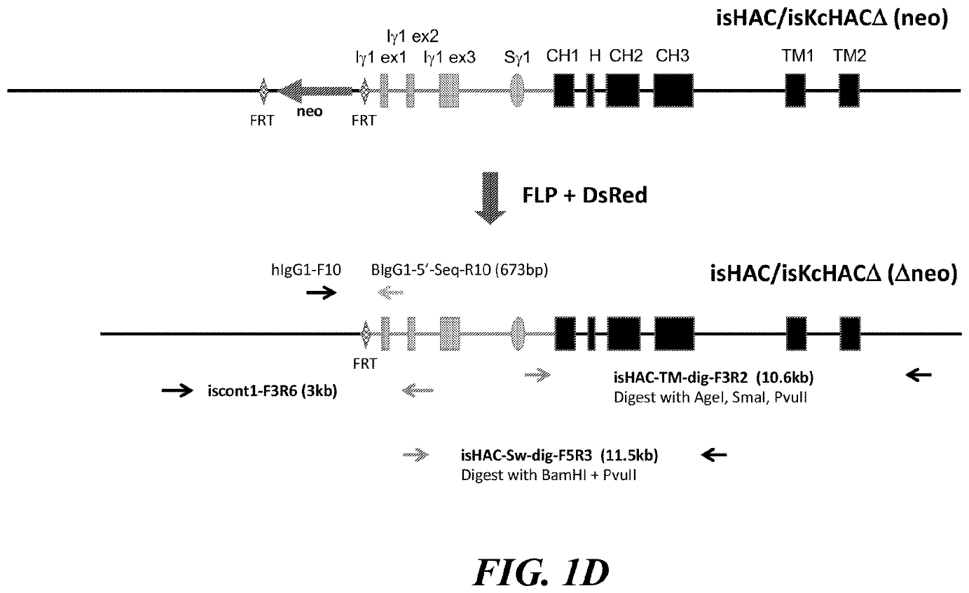

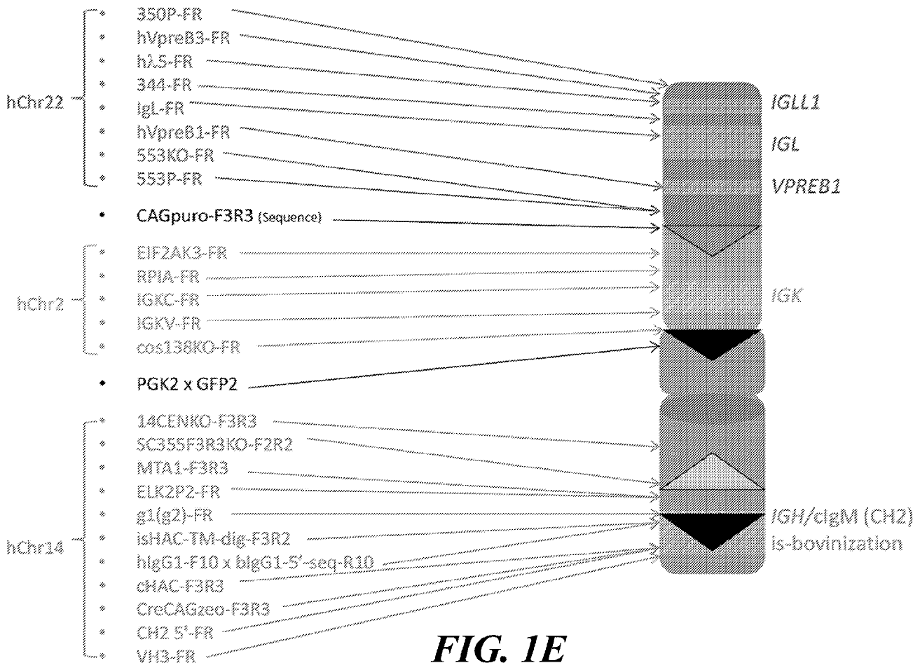

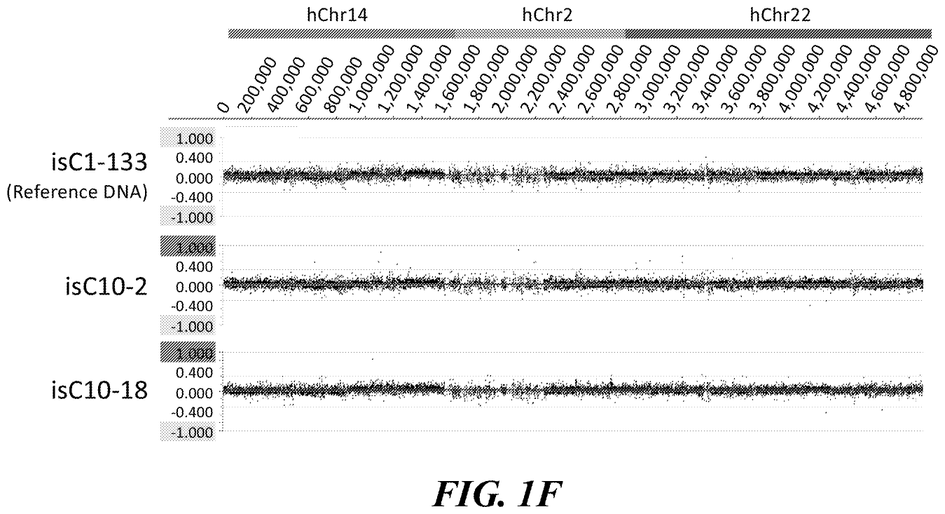

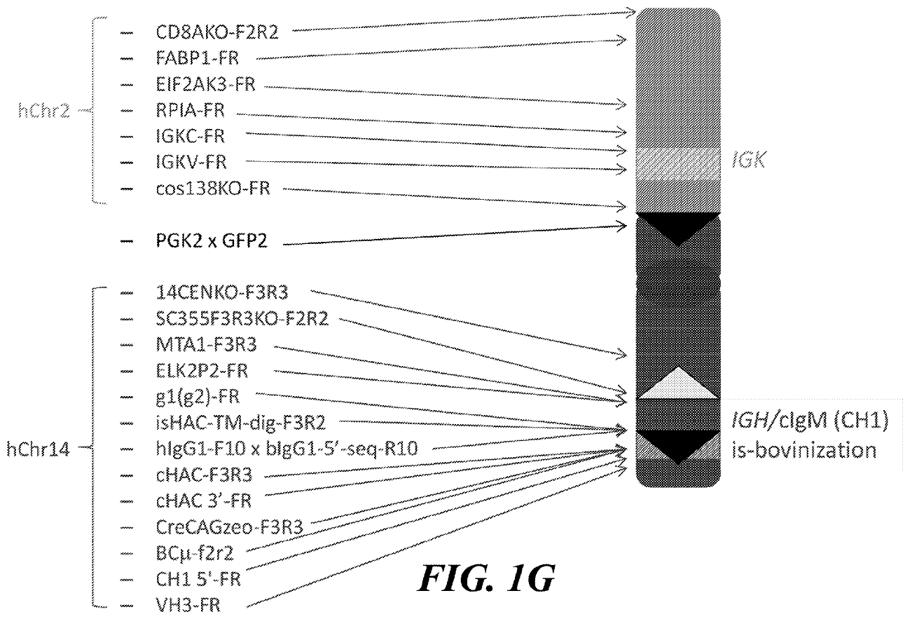

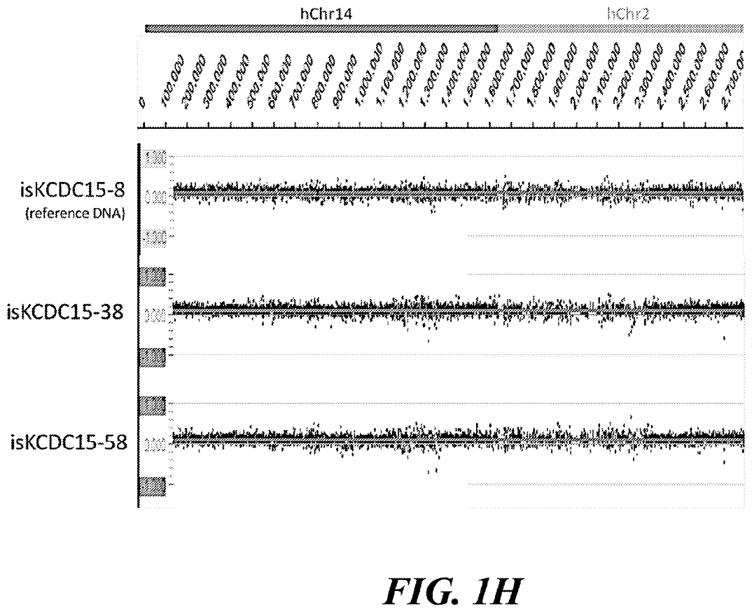

### DETAILED DESCRIPTION

The present inventors have developed a human immunoglobulin product for human disease that overcomes limitations of monoclonal antibody therapy. Transgenic animals with the endogenous Ig locus replaced by a human artificial chromosome encoding a human Ig locus express fully human polyclonal antibodies. Immunization of such a transgenic animal with a recombinant EGFR protein, or an antigenic fragment thereof, and/or with a polynucleotide encoding the antigen, generates polyclonal immunoglobulin with yield, purity, and antigen specificity that enable use of this product in medical applications. Various embodiments of the invention are provided in the description that follows.

### Definitions

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” “below,” and words of similar import, when used in this application, shall refer to this application as a whole and not to any particular portions of the application.

The term “ungulate” refers to any suitable ungulate, including but not limited to bovine, pig, horse, donkey, zebra, deer, oxen, goats, sheep, and antelope.

The term “transgenic” means the cells of the ungulate comprise one or more polynucleotides encoding exogenous gene(s) (e.g. an immunoglobulin locus). Such as polynucleotide may be a portion of an artificial chromosome. Alternatively, or in addition to an artificial chromosome, one or more polynucleotides encoding exogenous gene(s) may be integrated into the genome of the cells of the ungulate.

The terms “polyclonal” or “polyclonal serum” or “polyclonal plasma” or “polyclonal immunoglobulin” refer to a population of immunoglobulins having shared constant regions but diverse variable regions. The term polyclonal does not, however, exclude immunoglobulins derived from a single B cell precursor or single recombination event, as may be the case when a dominant immune response is generated. A polyclonal serum or plasma contains soluble forms (e.g., IgG) of the population of immunoglobulins. The term “purified polyclonal immunoglobulin” refers to polyclonal immunoglobulin purified by serum or plasma. Methods of purifying polyclonal immunoglobulin include, without limitation, caprylic acid fractionation and adsorption with red blood cells (RBCs).

A “population” of immunoglobulins refers to immunoglobulins having diverse sequences, as opposed to a sample having multiple copies of a single immunoglobulin. Similarly stated, the term population excludes immunoglobulins secreted from a single B cell, plasma cell, or hybridoma in culture, or from a host cells transduced or transformed with recombinant polynucleotide(s) encoding a single pair of heavy and light chain immunoglobulin sequences.

The term “immunoglobulin” refers to a protein complex of at least two heavy and at least two light chains in 1:1 ratio, including any of the five classes of immunoglobulin—IgM, IgG, IgA, IgD, IgE. In variations, the immunoglobulin is engineered in any of various ways known in the art or prospectively discovered, including, without limitation, mutations to change glycosylation patterns and/or to increase or decrease complement dependent cytotoxicity.

An immunoglobulin is “fully human or substantially human” when the protein sequence of the immunoglobulin is sufficiently similar to the sequence of a native human immunoglobulin that, when administered to a subject, the immunoglobulin generates an anti-immunoglobulin immune response similar to, or not significantly worse, that the immune reaction to native human immunoglobulin. A fully human immunoglobulin will comprise one or more substitutions, insertions, to deletions in variable regions, consistent with recombination, selection, and affinity maturation of the immunoglobulin sequence. In variations, the fully human or substantially human immunoglobulin is engineered in any of various ways known in the art or prospectively discovered, including, without limitation, mutations to change glycosylation patterns and/or to increase or decrease complement dependent cytotoxicity.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. Any of the compositions of the present disclosure may be isolated compositions.

The percentage of an immunoglobulin (e.g., immunoglobulin that specifically binds EGFR) “by mass of total immunoglobulin” refers to the concentration of a target immunoglobulin population divided by the concentration of total immunoglobulin in a sample, multiplied by 100. The concentration of target immunoglobulin can be determined by, for example, affinity purification of target immunoglobulin (e.g. on affinity column comprising EGFR) followed by concentration determination.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

The terms “immunization” and “immunizing” refer to administering a composition to a subject (e.g., a transgenic ungulate) in an amount sufficient to elicit, after one or more administering steps, a desired immune response (e.g., a polyclonal immunoglobulin response specific to EGFR). Administration may be by intramuscular injection, intravenous injection, intraperitoneal injection, or any other suitable route. Immunization may comprise between one and ten, or more administrations (e.g. injections) of the composition, such as 1, 2, 3, 4, 5, 6, 7, 8, 9, 10, or more administrations. The first administration may elicit no detectable immune response as generally each subsequence administration will boost the immune response generated by prior administrations.

The term “target antigen” refers to any antigen use to elicit a desired immune response. The target antigen used to generate an immunoglobulin product may be recombinant EGFR or an antigenic fragment thereof, or nucleic acid that encodes such proteins (e.g. RNA, linear DNA, or plasmid DNA).

The term “purify” refers to separating a target cell or molecule (e.g. a population of immunoglobulins) from other substances present in a composition. Immunoglobulins may be purified by fractionation of plasma, by affinity (e.g. protein A or protein G binding, or other capture molecule), by charge (e.g. ion-exchange chromatography), by size (e.g. size exclusion chromatograph), or otherwise. Purifying a population of immunoglobulins may comprise treating a composition comprising the population of immunoglobulins with one or more of acids, bases, salts, enzymes, heat, cold, coagulation factors, or other suitable agents. Purifying may further include adsorption of a composition comprising a target cell or molecule and an impurity onto non-target cells or molecules (e.g., red blood cells) to partially or completely remove the impurity. Purifying may further include pre-treatment of serum or plasma, e.g., caprylic acid fractionation.

The terms “treating” and “treatment” refer to one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “pharmaceutically acceptable” means biologically or pharmacologically compatible for in vivo use in animals or humans, and can mean approved by a regulatory agency of the Federal or a state government or listed in the U.S. Pharmacopeia or other generally recognized pharmacopeia for use in animals, and more particularly in humans.

The term “hyperimmunized” refers to immunization regimen that generates an immune response to the subject greater than required to produce a desired titer (e.g. a binding titer) after dilution of the immunoglobulin produced by the subject. For example, if a desired titer is 1:100, one may hyperimmunize an animal by a prime immunization followed by one, two, three or more boost immunizations to produce a 1:1,000 titer, or greater titer, in the subject—so that immunoglobulin produced by the subject may be diluted in the production of a biotherapeutic in order to give a desired titer in the biotherapeutic.

The term “affinity” refers to the strength of the interaction between an epitope and an antibody's antigen binding site. The affinity can be determined, for example, using the equation

KA=[Ab-Ag]/[Ab][Ag]

Where KA=affinity constant; [Ab]=molar concentration of unoccupied binding sites on the antibody; [Ag]=molar concentration of unoccupied binding sites on the antigen; and [Ab-Ag]=molar concentration of the antibody-antigen complex. The K_{A} describes how much antibody-antigen complex exists at the point when equilibrium is reached. The time taken for this to occur depends on rate of diffusion and is similar for every antibody. However, high-affinity antibodies will bind a greater amount of antigen in a shorter period of time than low-affinity antibodies. The K_{A} of the antibodies produced can vary and range from between about 10⁵ mol⁻¹ to about 10¹² mol⁻¹ or more. The K_{A} can be influenced by factors including pH, temperature, and buffer composition.

The antibody affinity can be measured using any means commonly employed in the art, including but not limited to the use of biosensors, such as surface plasmon resonance (SPR). Resonance units are proportional to the degree of binding of soluble ligand to the immobilized receptor (or soluble antibody to immobilized antigen). Determining the amount of binding at equilibrium with different known concentrations of receptor (antibody) and ligand (protein antigen) allows the calculation of equilibrium constants (K_{A}, K_{D}), and the rates of dissociation and association (k_{off}, kₒₙ).

The term “avidity” refers to the accumulated strength of multiple affinities of individual non-covalent binding interactions, such as between an antibody and its antigen. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. Avidity is measured by the off rate (k_{off}).

For example, KD (the equilibrium dissociation constant) is a ratio of k_{off}/kₒₙ, between the antibody and its antigen. KD and affinity are inversely related. The lower the KD value (lower antibody concentration), the higher the affinity of the antibody. Most antibodies have KD values in the low micromolar (10⁻⁶) to nanomolar (10⁻⁷ to 10⁻⁹) range. High affinity antibodies are generally considered to be in the low nanomolar range (10⁻⁹) with very high affinity antibodies being in the picomolar (10⁻¹²) range or lower (e.g. 10⁻¹³ to 10⁻¹⁴ range). In one embodiment, the antibodies produced by immunization with the EGFR-hFc antigen disclosed herein have a KD ranging from about 10⁻⁶ to about 10⁻¹⁵, from about 10⁻⁷ to about 10⁻¹⁵, from about 10⁻⁸ to about 10⁻¹⁵, and from about 10⁻⁹ to about 10⁻¹⁵, from about 10⁻¹⁰ to about 10⁻¹⁵, about 10⁻¹¹ to about 10⁻¹⁵, ab out 10⁻¹² to about 10⁻¹⁵, about 10⁻¹³ to about 10⁻¹⁴, about 10⁻¹³ to about 10⁻¹⁵, and about 10⁻¹⁴ to about 10⁻¹⁵.

The population of human immunoglobulins produced by the methods disclosed herein have high avidity, indicating they bind tightly to the antigen. In one embodiment, the antibodies produced by immunization with the EGFR-hFc antigen disclosed herein have an avidity ranging from about 10⁻¹ 1/sec to about 10⁻¹³ 1/sec, from about 10⁻³ 1/sec to about 10⁻¹³ 1/sec, from about 10⁻⁵ 1/sec to about 10⁻¹³ 1/sec, from about 10⁻⁶ 1/sec to about 10⁻¹³ 1/sec, from about 10⁻⁷ 1/sec to about 10⁻¹³ 1/sec, from about 10⁻⁸ 1/sec to about 10⁻¹³ 1/sec, from about 10⁻⁹ 1/sec to about 10⁻¹³ 1/sec, from about 10⁻¹⁰ 1/sec to about 10⁻¹³ 1/sec, from about 10⁻¹¹ 1/sec to about 10⁻¹³ 1/sec, or from about 10^{-12 1/}sec to about 10⁻¹³ 1/sec.

An immunoglobulin is “specific to” or “specifically binds” (used interchangeably herein) to a target (e.g., EGFR) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An immunoglobulin “specifically binds” to a particular protein or substance if it binds with greater affinity, avidity, more readily, and/or with greater duration than it binds to alternative particular protein or substance. For example, an immunoglobulin that specifically or preferentially binds to EGFR is an immunoglobulin that binds EGFR with greater affinity, avidity, more readily, and/or with greater duration than it binds to other proteins. An immunoglobulin that specifically binds to a first protein or substance may or may not specifically or preferentially bind to a protein (e.g., a member of the ErbB family of receptor tyrosine kinases), cell, or substance. As such, “specific binding” does not necessarily require (although it can include) exclusive binding. Generally, but not necessarily, reference to binding means specific binding.

The term “HAC vector” means a vector which comprises at least a human chromosome-derived centromere sequence, a telomere sequence, and a replication origin, and may contain any other sequences as desired for a given application. When present in a host cell, the HAC vector exists independently from a host cell chromosome in the nucleus. Any suitable methods can be used to prepare HAC vectors and to insert nucleic acids of interest into the HAC, including but not limited to those described in the examples that follow. The HAC vector is a double stranded DNA vector, as is known to those of skill in the art.

### Embodiments

Provided are methods of making a human polyclonal immunoglobulin for treatment of cancer, comprising administering an antigen comprising a EGFR or antigenic fragment thereof, or a polynucleotide encoding the antigen, to a transgenic ungulate, wherein the transgenic ungulate comprises a genome comprising a human immunoglobulin locus or an artificial chromosome comprising a human immunoglobulin locus, wherein the transgenic ungulate produces a population of human immunoglobulins that specifically binds the EGFR.

In a variation, non-human EGFR, or a polynucleotide encoding it, is used (e.g., a domesticated animal such as a dog, cat, sheep, etc.). The transgenic ungulate may in such cases comprise an artificial chromosome encoding an Ig locus of the non-human species such that antibodies of that species are generated.

In some embodiments, the EGFR, or a polynucleotide encoding it (that is, “the antigen”) is administered before, during, or after administration of one or more adjuvants. In some embodiments, the antigen and one or more adjuvants are administered together in a single composition, comprising optionally one or more pharmaceutically acceptable excipients.

Illustrative adjuvants include an aluminum salt adjuvant, an oil in water emulsion (e.g. an oil-in-water emulsion comprising squalene, such as MF59 or AS03), a TLR7 agonist (such as imidazoquinoline or imiquimod), or a combination thereof. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. Further illustrative adjuvants include, but are not limited to, Adju-Phos, Adjumerlm, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, MONTANIDE™ ISA-25, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant E1 12K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, ISA-25/Quil-A, Rehydragel HPA, Rehydragel LV, RIBI, Ribilike adjuvant system (MPL, TMD, CWS), S-28463, SAB-adj-1, SAB-adj-2, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes.

The immunization may be carried out by administering the antigen with, for example, a complete Freund's adjuvant or an appropriate adjuvant such as an aluminum hydroxide gel, and pertussis bacteria vaccine, subcutaneously, intravenously, or intraperitoneally into a transgenic ungulate. In one embodiment, the immunization comprises hyperimmunization. In various embodiments, the antigen is administered once to 10 times every 1 to 4 weeks after the first administration. After 1 to 14 days from each administration, blood is collected from the animal to measure the antibody value of the serum.

In some embodiments, the antigen is administered 3, 4, 5, 6 or more times. Administration of the EGFR may be performed, e.g., every 1-2 weeks, 2-3 weeks, 3-4 weeks, 4-5 weeks, 5-6 weeks, or 6-7 weeks, or longer intervals, e.g., every 1 week, 2 weeks, 3 weeks, 4 weeks, 5 weeks or 6 weeks. After each immunization, serum and/or plasma may be harvested from the transgenic ungulate one or more times. For example, the method may be including performing controls bleeds two or three times at intervals about 7-14 days.

In embodiments of the methods of the disclosure, the genome of the transgenic ungulate comprises a human immunoglobulin locus. Illustrative methods are provided in U.S. Pat. Nos. 9,902,970; 9,315,824; 7,652,192; and 7,429,690; and 7,253,334, the disclosure of which are incorporated by reference herein for all purposes. Further illustrative methods are provided by Kuroiwa, Y., et al. (2009) Nat Biotechnol. 27(2):173-81, and Matsushita et al. (2015) PLoS ONE 10(6):e0130699.

The disclosure provides a human artificial chromosome (HAC) vector comprising genes encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;
wherein at least one class switch regulatory element of the genes encoding the one or more human antibody heavy chains is replaced with an ungulate-derived class switch regulatory element.

The HAC vectors of the disclosure can be used, for example, for large-scale production of fully human antibodies by transgenic animals, as described for the methods of the invention. The HAC vector of the present disclosure comprises one or more genes encoding a human antibody heavy chain. Any human antibody heavy chain or combinations of human antibody heavy chains in combination may be encoded by one or more nucleic acids on the HAC. In various embodiments, 1, 2, 3, 4, 5, 6, 7, 8, or all 9 of human antibody heavy chains IgM, IgG1, IgG2, IgG3, IgG4, IgA1, IgA2, IgE and IgD may be encoded on the HAC in one or more copies. In one embodiment, the HAC comprises a human IgM antibody heavy chain encoding gene, alone or in combinations with 1, 2, 3, 4, 5, 6, 7, or the other 8 human antibody chain encoding genes. In one preferred embodiment, the HAC comprises a gene encoding at least a human IgG1 antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another embodiment, the HAC comprises a gene encoding at least a human IgA antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another preferred embodiment, the HAC comprises genes encoding all 9 antibody heavy chains, and more preferably where the gene encoding a human IgM antibody heavy chain has been chimerized to encode an ungulate-derived IgM heavy chain constant region. In another embodiment, the HAC may comprise a portion of human chromosome 14 that encodes the human antibody heavy chains. The variable region genes and the constant region genes of the human antibody heavy chain form a cluster and the human heavy chain locus is positioned at 14q32 on human chromosome 14. In one embodiment, the region of human chromosome 14 inserted in the HAC comprises the variable region and the constant region of the human antibody heavy chains from the 14q32 region of human chromosome 14.

In some embodiments of the HAC vectors of the present disclosure, at least one class switch regulatory element of the human antibody heavy chain encoding nucleic acid is replaced with an ungulate-derived class switch regulatory element. The class switch regulatory element refers to nucleic acid which is 5′ to an antibody heavy chain constant region. Each heavy chain constant region gene is operatively linked with (i.e. under control of) its own switch region, which is also associated with its own I-exons. Class switch regulatory elements regulate class switch recombination and determine Ig heavy chain isotype. Germline transcription of each heavy chain isotype is driven by the promoter/enhancer elements located just 5′ of the I-exons and those elements are cytokine or other activator-responsive. In a simple model of class switch, the specific activators and/or cytokines induce each heavy chain isotype germline transcription from its class switch regulatory element (i.e., activator/cytokine-responsive promoter and/or enhancer). Class switch is preceded by transcription of I-exons from each Ig heavy (IGH) locus-associated switch region. As each heavy chain constant region gene is linked with its own switch region.

Any suitable ungulate-derived class switch regulatory element can be used. For example, the human heavy chain gene isotypes listed below has the following class switch regulatory elements:
IgM: Iμ-Sμ
IgG1: Iγ1-Sγ1,
IgG2: Iγ2-Sγ2,
IgG3: Iγ3-Sγ3,
IgG4: Iγ4-Sγ4,
IgA1: Iα1-Sα1,
IgA2: Iα2-Sα2, and
IgE: Iε-Sε.

In various embodiments, 1, more than 1, or all of the human antibody heavy chain genes on the HAC have their class switch regulatory element replaced with an ungulate-derived class switch regulatory element, including but not limited to ungulate Iμ-Sμ, Iγ-Sγ, Iα-Sα, or Iε-Sε, class switch regulatory elements. In one embodiment, an Iγ1-Sγ1 human class switch regulatory element for human IgG1 heavy chain encoding nucleic acid on the HAC (such as that in SEQ ID NO: 1) is replaced with an ungulate Iγ1-Sγ1 class switch regulatory element. Exemplary ungulate Iγ1-Sγ1 class regulatory switch elements include a bovine IgG1 Iγ1-Sγ1 class switch regulatory element (SEQ ID NO: 2), a horse Iγ1-Sγ1 class switch regulatory element (SEQ ID NO: 3), and a pig Iγ1-Sγ1 class switch regulatory element (SEQ ID: 4). However, it is not necessary to replace the human class switch regulatory element with an ungulate class switch regulatory element from the corresponding heavy chain isotype. Thus, for example, an Iγ3-Sγ3 human class switch regulatory element for human IgG3 heavy chain encoding nucleic acid on the HAC can be replaced with an ungulate Iγ1-Sγ1 class switch regulatory element. As will be apparent to those of skill in the art based on the teachings herein, any such combination can be used in the HACs of the disclosure.

In another embodiment, the HAC comprises at least one ungulate enhancer element to replace an enhancer element associated with one or more human antibody heavy chain constant region encoding nucleic acids on the HAC. There are two 3′ enhancer regions (Alpha 1 and Alpha 2) associated with human antibody heavy chain genes. Enhancer elements are 3′ to the heavy chain constant region and also help regulate class switch. Any suitable ungulate enhancer can be used, including but not limited to 3′Eα enhancers. Non-limiting examples of 3′ Eα enhancers that can be used include 3′Eα, 3′Eα1, and 3′Eα2. Exemplary 3′Eα enhancer elements from bovine that can be used in the HACs and replace the human enhancer include, but are not limited to bovine HS3 enhancer (SEQ ID NO: 5), bovine HS12 enhancer (SEQ ID NO: 6), and bovine enhancer HS4. This embodiment is particularly preferred in embodiments wherein the HAC comprises the variable region and the constant region of the human antibody heavy chains from the 14q32 region of human chromosome 14.

The HAC vectors of the present disclosure may comprise one or more genes encoding a human antibody light chain. Any suitable human antibody light chain-encoding genes can be used in the HAC vectors of the invention. The human antibody light chain includes two types of genes, i.e., the kappa/K chain gene and the lambda/L chain gene. In one embodiment, the HAC comprises genes encoding both kappa and lambda, in one or more copies. The variable region and constant region of the kappa chain are positioned at 2p11.2-2p12 of the human chromosome 2, and the lambda chain forms a cluster positioned at 22q11.2 of the human chromosome 22. Therefore, in one embodiment, the HAC vectors of the invention comprise a human chromosome 2 fragment containing the kappa chain gene cluster of the 2p11.2-2p12 region. In another embodiment, the HAC vectors of the present invention comprise a human chromosome 22 fragment containing the lambda chain gene cluster of the 22q11.2 region.

In another embodiment, the HAC vector comprises at least one gene encoding a human antibody surrogate light chain. The gene encoding a human antibody surrogate light chain refers to a gene encoding a transient antibody light chain which is associated with an antibody heavy chain produced by a gene reconstitution in the human pro-B cell to constitute the pre-B cell receptor (preBCR). Any suitable human antibody surrogate light chain encoding gene can be used, including but not limited to the VpreB1 (SEQ ID NO: 7), VpreB3 (SEQ ID NO: 8) and λ5 (also known as IgLL1, SEQ ID NO: 9) human antibody surrogate light chains, and combinations thereof. The VpreB gene and the λ5 gene are positioned within the human antibody lambda chain gene locus at 22q11.2 of the human chromosome 22. Therefore, in one embodiment the HAC may comprise the 22q11.2 region of human chromosome 22 containing the VpreB gene and the λ5 gene. The human VpreB gene of the present invention provides either or both of the VpreB1 gene (SEQ ID NO: 7) and the VpreB3 (SEQ ID NO: 8) gene and in one embodiment provides both of the VpreB1 gene and the VpreB3 gene.

In yet another embodiment, the HAC vector comprises a gene encoding an ungulate-derived IgM heavy chain constant region. In this embodiment, the IgM heavy chain constant region is expressed as a chimera with the human IgM antibody heavy chain variable region. Any suitable ungulate IgM heavy chain antibody constant region encoding nucleic acid can be used, including but not limited to bovine IgM, (SEQ ID NO: 10), horse IgM, (SEQ ID NO: 11), sheep IgM, (SEQ ID NO: 12), and pig IgM, (SEQ ID NO: 13). In one embodiment, the chimeric IgM comprises the sequence in SEQ ID NO: 14. Pre-BCR/BCR signaling through the IgM heavy chain molecule promotes proliferation and development of the B cell by interacting with the B cell membrane molecule Ig-alpha/Ig-beta to cause a signal transduction in cells. Transmembrane region and/or other constant region of IgM are considered to have important roles in the interaction with Ig-alpha/Ig-beta for signal transduction. Examples of the IgM heavy chain constant regions include nucleic acids encoding constant region domains such as CH1, CH2, CH3, and CH4, and the B-cell transmembrane and cytoplasmic domains such as TM1 and TM2. The nucleic acid encoding an ungulate-derived IgM heavy chain constant region which is comprised in the human artificial chromosome vector of the invention is not particularly limited so long as the region is in a range which may sufficiently induce the signal of the B-cell receptor or B-cell proliferation/development in the above-described IgM heavy chain constant region. In one embodiment, the nucleic acid encoding an ungulate-derived IgM heavy chain constant region provides a transmembrane and cytoplasmic TM1 domain and TM2 domain derived from an ungulate, and in other embodiments encodes the ungulate-derived CH2 domain, CH3 domain, CH4 domain, TM1 domain, and TM2 domain or the ungulate-derived CH1 domain, CH2 domain, CH3 domain, CH4 domain, TM1 domain, and TM2 domain.

In one embodiment, the gene encoding the IgM heavy chain constant region of the bovine is a gene encoding a bovine IgM heavy chain constant region which is included in an IGHM region at which a bovine endogenous IgM heavy chain gene is positioned (derived from IGHM) or a gene encoding a bovine IgM heavy chain constant region in an IGHML1 region (derived from IGHML1). In another embodiment, the gene encoding a bovine IgM heavy chain constant region is included in the IGHM region.

In a further embodiment, the HAC comprises a gene encoding a human antibody heavy chain comprises a gene encoding a human heavy chain (for example, a human IgG heavy chain, such as an IgG1 heavy chain), and wherein a transmembrane domain and an intracellular domain of a constant region of the human heavy chain gene are replaced with a transmembrane domain and an intracellular domain of an ungulate-derived heavy chain (for example, an ungulate IgG heavy chain, such as an IgG1 heavy chain), constant region gene. In one embodiment, gene encoding the transmembrane domain and the intracellular domain of an ungulate-derived (such as bovine) IgG (such as IgG1) heavy chain constant region are used to replace the corresponding regions of the human IgG heavy chain gene. In another embodiment, the gene encoding the TM1 and TM2 domains of an ungulate-derived (such as bovine) IgG (such as IgG1) heavy chain constant region are used to replace the corresponding regions of the human IgG heavy chain gene. In another embodiment, the gene encoding the one or more of the CH1-CH4 domains and/or the TM1 and TM2 domains of an ungulate-derived (such as bovine) IgG (such as IgG1) heavy chain constant region are used to replace the corresponding regions of the human IgG heavy chain gene.

In one embodiment, avidity of a molecular interaction between two molecules can be measured via different techniques, such as the well the known surface plasmon resonance (SPR) biosensor technique where one molecule is immobilized on the biosensor chip and the other molecule is passed over the immobilized molecule under flow conditions yielding kₒₙ, k_{off} measurements and hence avidity values.

In one embodiment, the population of human immunoglobulins may be said to bind a target polypeptide disclosed herein or a fragment or variant thereof with an avidity of less than or equal to 10⁻¹ 1/sec, 10⁻² 1/sec, or 10⁻³ 1/sec. In one embodiment, the population of human immunoglobulins of the invention may be said to bind a target polypeptide disclosed herein or a fragment or variant thereof with an avidity less than or equal to 10⁻⁴ 1/sec, 10⁻⁵ 1/sec, 10⁻⁶ 1/sec, or 10⁻⁷ 1/sec.

In one embodiment, the population of human immunoglobulins comprises an avidity for EGFR of at least 120%, at least 110%, at least 100%, at least 90%, at least 80%, at least 70%, at least 60%, at least 50%, or at least 40%, that of ATCC Deposit No. PTA-127158.

The disclosure further provides transgenic ungulates comprising a HAC vector according to any embodiment or combination of embodiments of the disclosure. The transgenic ungulate comprising the HAC vector of the present invention refers to an animal into which the human artificial chromosome vector of the present invention is introduced. The transgenic ungulate having the HAC of the present invention is not particularly limited so long as the animal is a transgenic ungulate in which the human artificial chromosome fragment may be introduced into a cell thereof, and any non-human animals, for example, ungulates such as cows, horses, goats, sheep, and pigs; and the like may be used. In one aspect, the transgenic ungulate is a bovine. A transgenic ungulate having the HAC vector of the present invention may be constructed, for example, by introducing the HAC vector of the present disclosure into an oocyte of a host animal using any suitable technique, such as those described herein. The HAC vector of the present invention may, for example, be introduced into a somatic cell derived from a host ungulate by a microcell fusion method. Thereafter, the animal having the HAC vector may be constructed by transplanting a nucleus or chromatin agglomerate of the cell into an oocyte and transplanting the oocyte or an embryo to be formed from the oocyte into the uterus of a host animal to give birth. It may be confirmed by a method of Kuroiwa et al. (Kuroiwa et al., Nature Biotechnology, 18, 1086-1090, 2000 and Kuroiwa et al., Nature Biotechnology, 20, 889-894) whether an animal constructed by the above method has the human artificial chromosome vector.

The disclosure further provides transgenic ungulates comprising genes integrated into its genome encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;
wherein at least one class switch regulatory element of the genes encoding the one or more human antibody heavy chains is replaced with an ungulate-derived class switch regulatory element.

In such embodiments, the transgenic ungulate may comprise any embodiment or combination of embodiments of the nucleic acids as described herein for the HAC, but rather than being present in a HAC, they are integrated into a chromosome of the ungulate.

The disclosure further provides a method of producing a human antibody, comprising: (a) administering EGFR, or other target antigen of the disclosure, to the transgenic ungulate of any embodiment or combination of embodiments of the disclosure to produce and accumulate a population of human immunoglobulins specific to EGFR (or T cells, B cells, and/or monocytes) in the serum or plasma of the ungulate; and optionally (b) isolating, recovering, and/or purifying the population of human immunoglobulins specific to the EGFR (or T cells, B cells, and/or monocytes) from the serum or plasma of the ungulate.

The polyclonal serum or plasma, or human immunoglobulin purified from the polyclonal serum or plasma, may be used as an immunoglobulin product for cancer.

In a variation, the disclosure provides a method of recovering the protein sequence of a human antibody comprises: (i) isolating lymphocytes from the transgenic ungulate; (ii) generating a human monoclonal antibody producing hybridoma from the lymphocytes; and (iii) recovering human monoclonal antibody specific to the antigen from the hybridoma. In another embodiment, the lymphocytes from the transgenic ungulate are isolated from lymph nodes of the transgenic ungulate. In a further embodiment the transgenic ungulate is hyperimmunized with the target antigen.

A EGFR-specific human immunoglobulin (such as EGFR-specific human immunoglobulin) may be produced by immunizing the transgenic ungulate having the HAC vector with human EGFR, or another antigen of the disclosure, to produce the EGFR-specific human immunoglobulin in the serum or plasma of the transgenic ungulate and recovering the EGFR-specific human immunoglobulin from the serum or plasma of the transgenic ungulate.

Examples of methods for detecting and measuring the EGFR-specific human immunoglobulin in a composition include a binding assay by an enzyme-linked immunosorbent assay, and the like. The binding amount of a human immunoglobulin may be measured by incubating the composition comprising the human immunoglobulin with cells (e.g., T cells, B cells and/or monocytes, or recombinant protein antigen(s)), and then using an antibody specifically recognizing human immunoglobulin.

In a variation, the methods of the disclosure are used to generate a monoclonal antibody. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Kohler and Milstein, Nature 256:495 (1975)). An example of a preparation method for hybridomas comprises the following steps of: (1) immunizing a transgenic ungulate with a recombinant EGFR; (2) collecting antibody-producing cells from the transgenic ungulate (i.e. from lymph nodes); (3) fusing the antibody-producing cells with myeloma cells; (4) selecting hybridomas that produce a monoclonal antibody specific to EGFR from the fused cells obtained in the above step; and optionally (5) selecting a hybridoma that produces a monoclonal antibody specific to EGFR from the selected hybridomas.

In embodiments of the methods of producing polyclonal immunoglobulin specific for EGFR (such as EGFR-specific human immunoglobulin), the transgenic ungulate produces human polyclonal immunoglobulin specific for EGFR. The method may comprise collecting the polyclonal serum and/or polyclonal plasma from the transgenic ungulate. In some embodiments, the ungulate is a bovine. In some embodiments, the polyclonal immunoglobulin composition comprises a population of fully human immunoglobulins. In some embodiments, the polyclonal immunoglobulin composition comprises a population of fully human immunoglobulins, substantially human immunoglobulins.

Some embodiments of the methods of the disclosure, and related compositions, have the surprising advantage that the EGFR-specific immunoglobulins (such as EGFR-specific human immunoglobulin) are produced in high yield, in high purity, and/or as a high percentage of total immunoglobulin present in the serum or plasma of the transgenic ungulate. Furthermore, some embodiments produce EGFR-specific immunoglobulins having glycans that comprise at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95% or higher percentage of fucosylated glycans. Furthermore, some embodiments produce EGFR-specific immunoglobulins having at most about the same ADCC or CDC activity as a reference immunoglobulin preparation, e.g. human-derived immunoglobulin.

In some embodiments, the population of human immunoglobulins binds FcγRI with a K_{D} of 15 nM or greater. In some embodiments, the population of human immunoglobulins binds FcγRIIa with a K_{D} of 500 nM or greater. In some embodiments, the population of human immunoglobulins binds FcγRIIb/c with a K_{D} of 1 μM or greater. In some embodiments, the population of human immunoglobulins binds FcγRIIIa with a K_{D} of 1 μM or greater. In some embodiments, the population of human immunoglobulins binds FcγRIIIa with a K_{D} of 1 nM or greater.

In some embodiments of the methods and compositions of the disclosure, the polyclonal serum or polyclonal plasma comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 1-4%, 2-5%, 3-6%, 4-7%, 5-8%, 6-9%, or 7-10% fully human (or substantially human) immunoglobulin by mass of total immunoglobulin in the polyclonal serum or polyclonal plasma.

In some embodiments of the methods and compositions of the disclosure, the polyclonal immunoglobulin comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal immunoglobulin.

In some embodiments of the methods and compositions of the disclosure, the polyclonal immunoglobulin comprises 0-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal immunoglobulin.

In some embodiments of the methods and compositions of the disclosure, the polyclonal immunoglobulin comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 1-4%, 2-5%, 3-6%, 4-7%, 5-8%, 6-9%, or 7-10% fully human (or substantially human) immunoglobulin by mass of total immunoglobulin in the polyclonal immunoglobulin.

In some embodiments of the methods and compositions of the disclosure, the polyclonal immunoglobulin comprises at least 5% fully human immunoglobulin by mass of total immunoglobulin in the polyclonal immunoglobulin.

In some embodiments of the methods and compositions of the disclosure, the polyclonal immunoglobulin comprises 2% to 5% fully human immunoglobulin by mass of total immunoglobulin in the polyclonal immunoglobulin.

In some embodiments, the ungulate-derived polyclonal immunoglobulin comprises “chimeric” human immunoglobulin having a human heavy chain and an ungulate kappa light chain (termed “cIgG”). In some embodiments, the polyclonal immunoglobulin comprises less than about 0.5%, less than about 0.75%, less than about 1.0%, less than about 1.25%, less than about 1.5%, less than about 1.75%, less than about 2.0%, less than about 2.25%, less than about 2.5%, less than about 2.75%, less than about 3.0%, less than about 3.25%, less than about 3.5%, less than about 3.75%, or less than about 4.0% cIgG as a percent of total protein concentration. In some embodiments, the polyclonal immunoglobulin comprises about 0.5% to about 1.0%, about 1.0% to about 1.5%, about 1.5% to about 2.0%, about 1.5% to about 2.0%, about 2.0% to about 2.5%, or about 2.5% to about 3.0% cIgG as a percent of total protein concentration. In some embodiments, the polyclonal immunoglobulin comprises about 0.5% to about 1.0%, about 1.0% to about 2.0%, or about 1.0 to about 3.0% cIgG as a percent of total protein concentration.

In some embodiments, the polyclonal immunoglobulins of the disclosure are less potent in a complement-dependent cytotoxicity (CDC) assay than a reference product (e.g. human-derived polyclonal immunoglobulin). In some embodiments, the polyclonal immunoglobulins of the disclosure are at most about 5%, at most about 10%, at most about 25%, at most about 50%, at most about 100%, at most about 150%, or more at most about 200% potent in a complement-dependent cytotoxicity (CDC) assay than a reference product (e.g. human-derived polyclonal immunoglobulin).

In some embodiments, the polyclonal immunoglobulins of the disclosure generate lower toxicity towards CD8+cells than a reference product (e.g. human-derived polyclonal immunoglobulin. In some embodiments, the polyclonal immunoglobulins of the disclosure are at most about 5%, at most about 10%, at most about 25%, at most about 50%, at most about 100%, at most about 150%, or at most about 200% more potent in CD8+cell killing assay than a reference product (e.g. human-derived polyclonal immunoglobulin).

In some embodiments, the polyclonal immunoglobulins of the disclosure generated lower rates of CD4+T cell apoptosis than a reference product (e.g. human-derived polyclonal immunoglobulin. In some embodiments, the polyclonal immunoglobulins of the disclosure are at least about 5%, at least about 10%, at least about 25%, at least about 50%, at least about 100%, at least about 150%, or at least about 200% less toxic in a CD4+cell apoptosis assay than a reference product (e.g. human-derived polyclonal immunoglobulin).

In some embodiments, the polyclonal immunoglobulins of the disclosure better preserves T_{reg} to conventional T cell rations than a reference product (e.g. human-derived polyclonal immunoglobulin. In some embodiments, the polyclonal immunoglobulins of the disclosure are at least about 5%, at least about 10%, at least about 25%, at least about 50%, at least about 100%, at least about 150%, or at least about 200% less toxic to T_{reg} cells than a reference product (e.g. human-derived polyclonal immunoglobulin).

In some embodiments of the methods and compositions of the disclosure, the population of fully human immunoglobulins (or substantially human) specifically binds EGFR, or an immunologically similar antigen.

In some embodiments, a genome of the transgenic ungulate comprises a human immunoglobulin locus.

In some embodiments, the transgenic ungulate is immunized 3, 4, 5, or more times.

In some embodiments, the population of fully human or substantially human immunoglobulins are purified from the serum of the transgenic ungulate after immunization.

The disclosure provides methods of providing human polyclonal immunoglobulin specific for EGFR (such as EGFR) treatment to a subject in need thereof, comprising administering to the subject a polyclonal immunoglobulin according to the disclosure. In some embodiments, the method provides an effective amount of human polyclonal immunoglobulin specific for EGFR to the subject.

The disclosure provides methods of providing human polyclonal immunoglobulin specific for EGFR (such as EGFR) treatment to a subject in need thereof, comprising administering to the subject a composition produced by immunizing a transgenic ungulate with EGFR. In some embodiments, the method provides an effective amount of human polyclonal immunoglobulin specific for EGFR to the subject.

The disclosure provides methods of providing human polyclonal immunoglobulin specific for EGFR (such as EGFR) treatment to a subject in need thereof, comprising administering to the subject a polyclonal immunoglobulin produced according to the disclosure. In some embodiments, the method provides an effective amount of human polyclonal immunoglobulin specific for EGFR to the subject.

The disclosure further provides pharmaceutical compositions, comprising a population of fully human or substantially human immunoglobulins, and one or more pharmaceutically acceptable excipients. In some embodiments, the population of fully human or substantially human immunoglobulins specifically binds human EGFR, or antigenic fragment thereof.

In some embodiments, the pharmaceutical composition comprises at least about 1 mg/mL, at least about 50 mg/mL, at least about 100 mg/mL, or at least about 1,000 mg/mL of fully human or substantially human immunoglobulin. In some embodiments, the pharmaceutical composition comprises at least about 100 μg/mL, at least about 250 μg/mL, at least about 500 μg/mL, at least about 750 μg/mL, or at least about 1,000 μg/mL of fully human or substantially human immunoglobulin.

In some embodiments, the fully human or substantially human immunoglobulin is produced in an ungulate. In some embodiments, the ungulate is a bovine.

In some embodiments, the pharmaceutical composition comprises at least 5% fully human immunoglobulin by mass of total immunoglobulin in the pharmaceutical composition.

In some embodiments, the pharmaceutical composition comprises 2% to 5% fully human immunoglobulin by mass of total immunoglobulin in the pharmaceutical composition.

### EXAMPLES

The following specific examples are to be construed as merely illustrative, and not limitative of the remainder of the disclosure in any way whatsoever.

### Example 1

### Characterization of Anti-EGFR Polyclonal Antibodies Generated in a Transchromosomic Bovine

Generation of Purified hEGFR-ECD

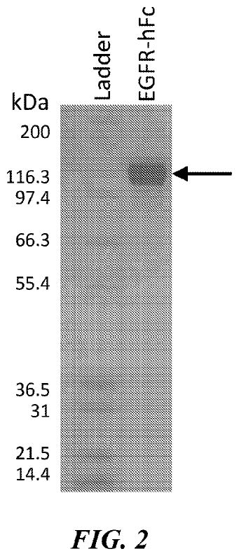

The Epithelial Growth Factor Receptor (EGFR), also known as ErbB1 or HER1, is a member of the ErbB family of receptor tyrosine kinases. EGFR mediated downstream cell signaling pathways control fundamental cellular functions including proliferation, survival and apoptosis. To produce anti-EGFR human polyclonal antibodies using the Tc bovine platform, an expression construct encoding EGFR-human Fc (hFc) antigen was generated. The pEGFR-hFc plasmid encodes the extracellular domain (ECD) of human EGFR comprised of amino acids 1-638. A thirteen amino acid linker connects the EGFR-ECD to amino acids 104-330 of the ImmunoGlobulin Heavy constant Gamma 1 (IGHG1) on the C-terminus of the fusion protein (SEQ ID NO: 16). The pEGFR-hFc was transfected into FreeStyle 293F suspension cells. EGFR-rFc was purified from the supernatant using an affinity chromatography purification column, and the protein was analyzed by SDS-PAGE to verify purity and size (FIG. 2). The predicted size of EGFR-hFc is 97.2 kDa. However, the protein runs at ˜116 kDa on an SDS-PAGE gel due to post-translational modifications such as glycosylation.

TABLE 1
Vaccination Interval and Formulation

| Tc Bovine | Vaccination | Vaccine Formulation |
|---|---|---|
| TKO/isKcHACΔ | V1 to V4 with | 2 mg EGFR-hFc + ISA-206/Quil A |
| | 3-week intervals | for V1 and V2 |
| | | 5 mg EGFR-hFc + ISA-206/Quil A |
| | | for V3 and V4 |

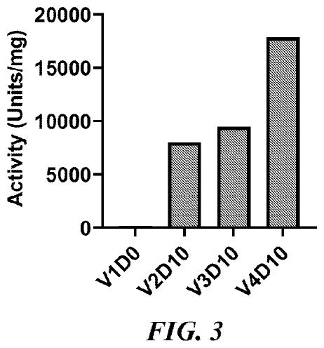

One Tc bovine was hyperimmunized with 2 mg of EGFR-hFc for vaccinations V1 and V2. Five mg of EGFR-hFc was administered per dose for V3 and V4 for a total of 4 vaccinations (Table 1). The Tc animal was immunized with EGFR-hFc vaccine via intramuscular injections on both sides of neck and hind leg regions with equal vaccine volume on each area. Serum was collected from the Tc bovine over the course of the four immunizations, and ELISA was performed to determine the titer against EGFR (FIG. 3). The titer of anti-EGFR antibodies increased over the course of vaccinations.

Collected plasma 14 days after the fourth vaccination (V4D14) was frozen for shipment, thawed, pooled, fractionated by caprylic acid (CA), and clarified by depth filtration. Clarified material containing Tc bovine-derived human immunoglobulin G (IgG) was purified by affinity chromatography using an anti-human IgG affinity column first to bind Tc bovine-derived human IgG (hIgG) and remove bovine plasma proteins (BPP). Second, a low pH treatment for viral inactivation is performed following by using an anti-bovine IgG (bIgG) heavy chain (HC) specific affinity column to further remove residual IgG molecules that contain a bovine HC or Fc of bovine HC. The Tc bovine-derived human IgG fraction was then concentrated and diafiltered prior to a Q Sepharose chromatography polishing step, nanofiltration, final buffer exchange, concentration and sterile filtration.

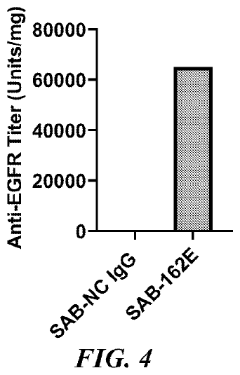

The purified human anti-EGFR polyclonal antibodies are referred to as SAB-162E. An indirect ELISA was performed on SAB-162E using Tc bovine pre-immune plasma as a negative control (FIG. 4). While the titer against EGFR of the SAB-NC IgG was 75 unit/mg, SAB-162E had a titer of 65,007 units/mg at V4D14.

Binding of TcB-Derived Anti-EGFR Polyclonal Antibodies to Cell Surface EGFR.

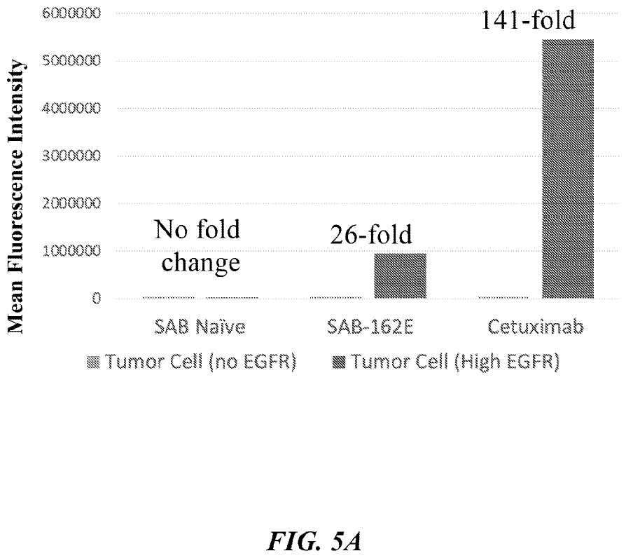

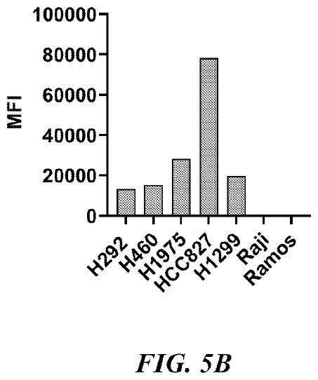

The binding of polyclonal antibodies to EGFR was assessed using the human leukemia cell line, HL-60, (EGFR negative) and breast cancer cell line, MDA-MB-468 (EGFR high). The antibodies tested for binding were cetuximab, a positive control anti-human EGFR antibody (Biolegend®), SAB-162E, and naïve TcB negative control polyclonal antibody. Cell lines were incubated with the fluorescently labeled primary antibodies, and flow cytometry analysis was performed (FIG. 5A). SAB-162E demonstrated targeted binding to EGFR-expressing MDA-MB-486 cells and no binding to HL-60, which is EGFR negative. Benchmarked against the commercial product cetuximab, SAB-162E has ˜5-fold lower binding to MDA-MB-486 at 10 μg/ml antibody concentration.

To further evaluate the binding of SAB-162E to EGFR-expressing cancer cell lines, human non-small cell lung carcinoma cell lines that were positive for EGFR on the cell surface were studied, including H292, H460, H1975, HCC827, and H1299. For a negative binding control, EGFR negative Raji and Ramos lymphoma cells were utilized. SAB-162E was directly conjugated to AF-488, and cell lines were incubated with the fluorescently labeled primary antibodies. Flow cytometry analysis was performed to determine the relative amount of surface EGFR by measuring fluorescent intensity levels of the live cells (FIG. 5B). SAB-162E demonstrated targeted binding to EGFR-expressing H292, H460, H1975, HCC827, and H1299 cells and no binding to Raji and Ramos lymphoma cells, which are EGFR negative. HCC827 had the highest level of EGFR followed by H1975, and H292 had the least amount of the EGFR target protein.

Titration of SAB-162E Binding to Human NSCLC Cell Lines

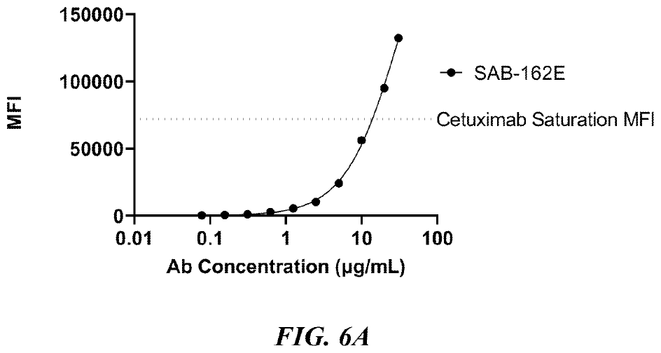

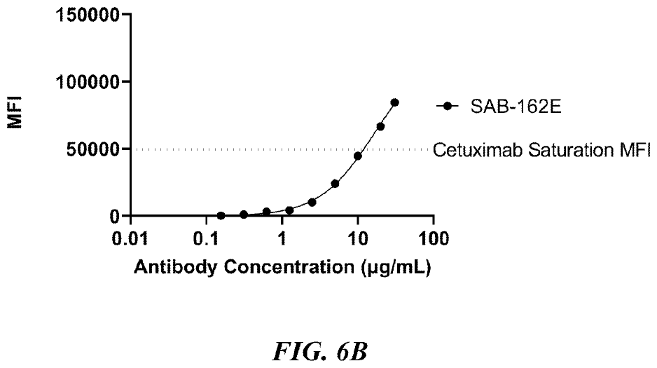

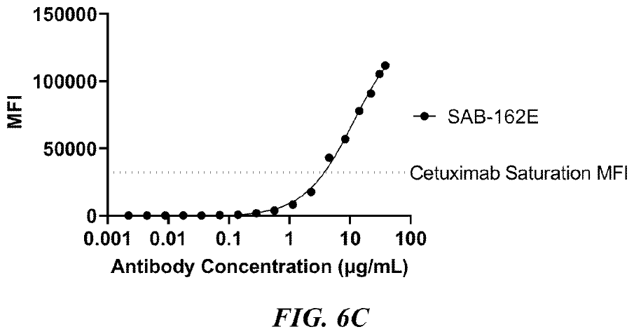

An SAB-162E titration assay was performed to determine the antibody concentration required for saturating binding to human NSCLC cells. For comparison, the saturating concentrations of cetuximab to the same cell lines were measured. The primary antibodies, SAB-162E and cetuximab, were directly conjugated to AF-488, and antibody serial dilutions were added to the cells. Saturating binding levels of SAB-162E and cetuximab were determined for three NSCLC cell lines, H292 (FIG. 6A), H1975 (FIG. 6B) and H1299 (FIG. 6C). Levels of SAB-162E continue to increase with increasing amount of added primary antibody. However, cetuximab reached saturation levels at a lower MFI than SAB-162E. This difference may be due to the polyclonal nature of SAB-162E where multiple antibodies bind to a single cell. Due to technical limitations of direct labeling of the primary antibody, a plateau in the graph indicating saturation levels for SAB-162E was not reached in the cell lines. This data indicates that SAB-162E binds to EGFR-expressing cells in a dose dependent manner.

Antibody-Dependent Cell-Mediated Cytotoxicity (ADCC) Activity of SAB-162E

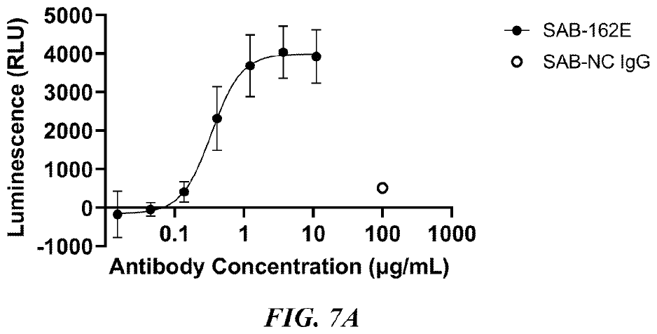

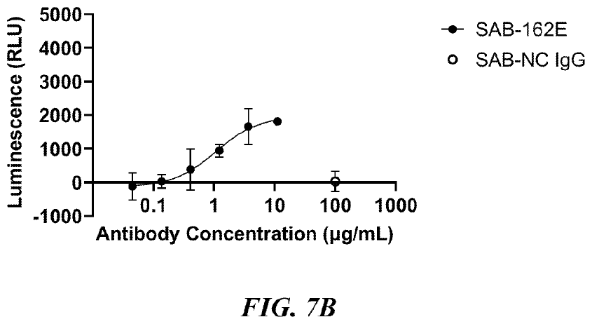

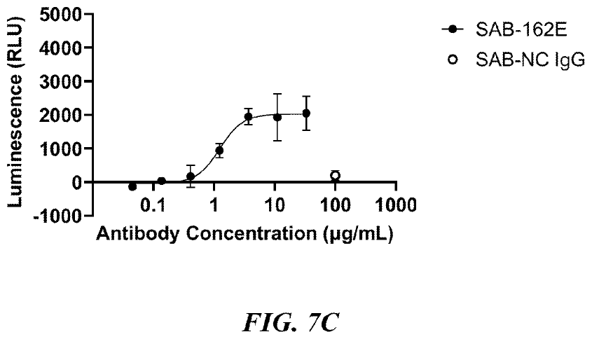

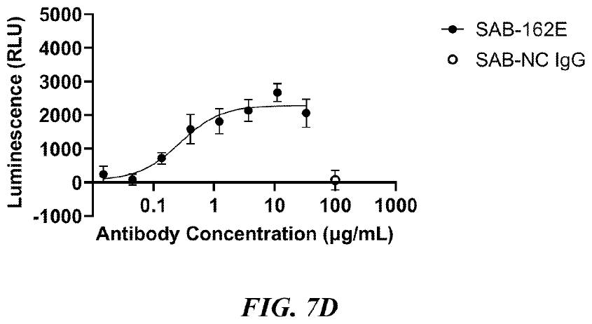

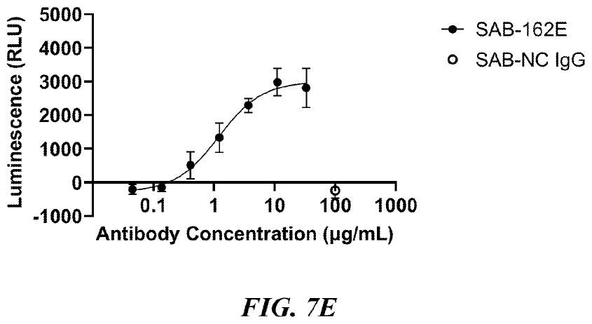

Antibody-dependent cell-mediated cytotoxicity (ADCC) is a mechanism by which antibodies target cancer cells for destruction by the cell-mediated immune system, such as natural killer (NK) cells. IgG antibodies bind to target antigens on cancer cells, and the Fc regions of the antibodies are recognized by FcγRIIIa (CD16) molecules on NK cells as well as macrophages. Upon binding of the IgG Fc to FcγRIIIa, a signal transduction cascade is initiated, and the nuclear factor of activated T cells (NFAT) mediates activation of cytokine genes. The InvivoGen Jurkat-Lucia™ NFAT-CD16 effector cells takes advantage of this NFAT pathway to create an ADCC reporter assay. Jurkat-Lucia™ NFAT-CD16 cells are human T lymphocyte cells that have been modified to stably express FcγRIIIa (CD16). Also, the cells stably express the Lucia luciferase reporter gene under the control of NFAT response elements. This assay gives a quantitative measurement of ADCC initiation by assessing cellular levels of luminescence. The human NSCLC cell lines, H1299, H460, H1975, H292 and HCC827, all have EGFR present on the cell surface (FIG. 5B) and were used as target cells for the assay. To determine the ability of SAB-162E to initiate ADCC, H1299 (FIG. 7A), H460 (FIG. 7B), H1975 (FIG. 7C), H292 (FIG. 7D) and HCC827 (FIG. 7E) cells were incubated with serial dilutions of SAB-162E. Naïve TcB negative control pAb was added to the cells only at the highest concentration. The effector cells, Jurkat-Lucia™ NFAT-CD16 cells, were added to the NSCLC target cells in the ratio of effector cell to target cell (E:T ratio) of 6:1. The plates were incubated for 24 to 48 hours, and luminescence was measured. Compared to the negative control pAb, SAB-162E demonstrated ADCC killing of all 5 NSCLC cells (FIG. 7). The highest level of luciferase occurred at ˜10 mg/mL.

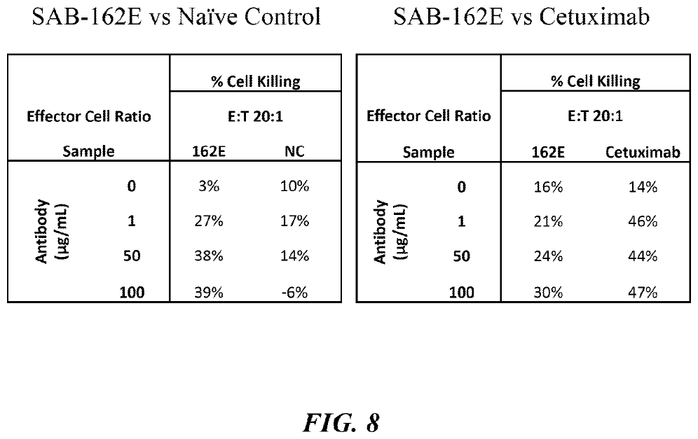

To further investigate ADCC activity of SAB-162E, MDA-MB-468 was used as the target cell for the assay. Three different antibodies were tested: cetuximab, SAB-162E, and naïve TcB negative control pAb. MDA-MB-468 cells were plated onto ACEA Biosciences 16-well plates and incubated overnight. The antibodies were added to the cells at a concentration of 0, 1, 50 and 100 μg/ml for 60 minutes. The effector cells, PBMCs, were added to MDA-MB-468 in the ratio of effector cell to target cell (E:T ratio) of 20:1, and the plates are incubated for 24 hours. The ACEA Bioscience plate reader operates in the cell culture incubator while the control unit is outside on the bench. The instrument uses biosensors to measure the impedance of the attached cells, and impedance is measured in units called Cell Index (CI). The instrument can monitor cell death in real time. Compared to the negative control pAb, SAB-162E demonstrated ADCC killing of the MDA-MB-486 cells (FIG. 8).

Complement-Dependent Cytotoxicity (CDC) Activity of SAB-162E

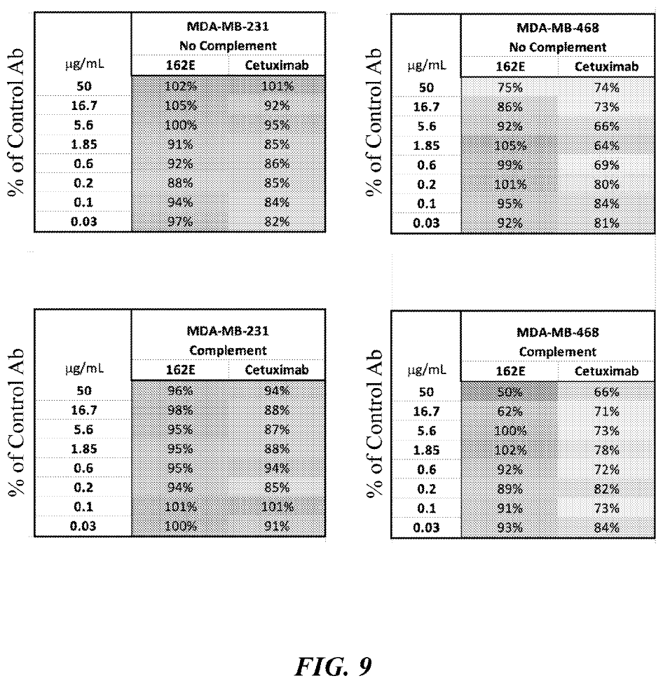

To measure the CDC activity of SAB-162E, MDA-MB-468 (EGFR high) and MDA-MB-231 (EGFR low) cell lines were used. Cultured cells were incubated with cetuximab, SAB-162E, or naïve TcB negative control pAb at dilutions ranging from 0.03 to 50 μg/ml. After a one-hour incubation, ice cold complement or complete media as a no complement control was added to the cells. The cells were incubated for 72 hours, and cell viability was measured using a CellTiter-Glo reagent. Results were calculated as a percentage of the negative control antibody. SAB-162E and cetuximab demonstrated similar targeted binding at the higher concentrations of antibody in the presence of complement (FIG. 9). Without the addition of complement, cetuximab inhibited cell proliferation of MDA-MB-231 cells, and SAB-162E showed growth inhibition at the highest concentration of 50 μg/ml. At 50 μg/ml, SAB-162E demonstrated 50% cell viability compared to the negative control pAb suggesting that SAB-162E has functional complement activity (FIG. 9).

SAB-162E Blocks EGF Binding to EGFR on Surface of NSCLC Cells

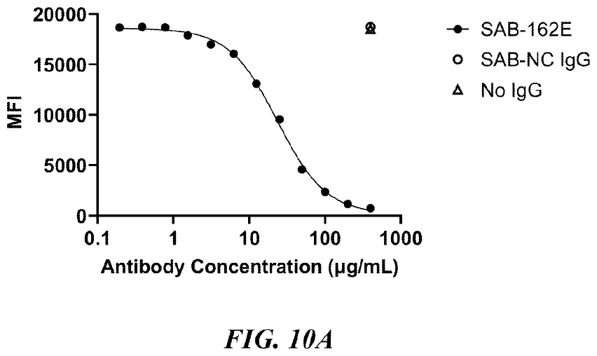

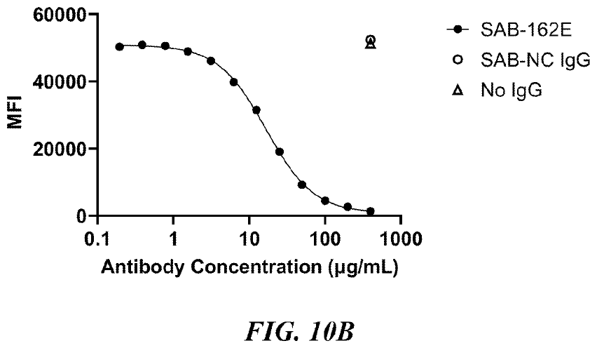

One mechanism by which antibody therapies increase the efficacy of cancer patients is to block ligand binding to the targeted receptor. By blocking ligand binding, the initiation of signal transduction pathways that result in cell proliferation is inhibited. A binding assay using NSCLC cells was performed to determine whether SAB-162E blocks the binding of EGF to EGFR. H1299 and H292 cells were pre-incubated with increasing amounts of SAB-162E. For controls, SAB-NC IgG or no antibody was added to the cells only at the highest concentration. Saturating levels of EGF-Alexa Fluor conjugate were added, and the mean fluorescence intensity (MFI) was measured by flow cytometry. SAB-162E completely blocked binding of EGF with an average IC₅₀ of 22 μg/mL for H1299 (FIG. 10A) and 43 μg/mL for H292 (FIG. 10B). Maximum binding levels were demonstrated by adding saturating amounts of EGF to the cells with no antibody. Blockage of EGF to the EGFR was specific for SAB-162E as indicated by MFI levels of SAB-NC IgG being similar to the level of no antibody added.

Effect of SAB-162 on Downstream EGFR Signaling

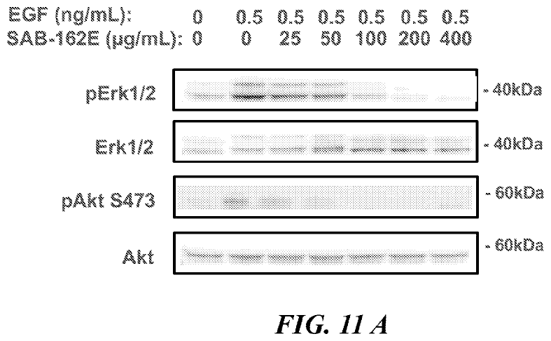

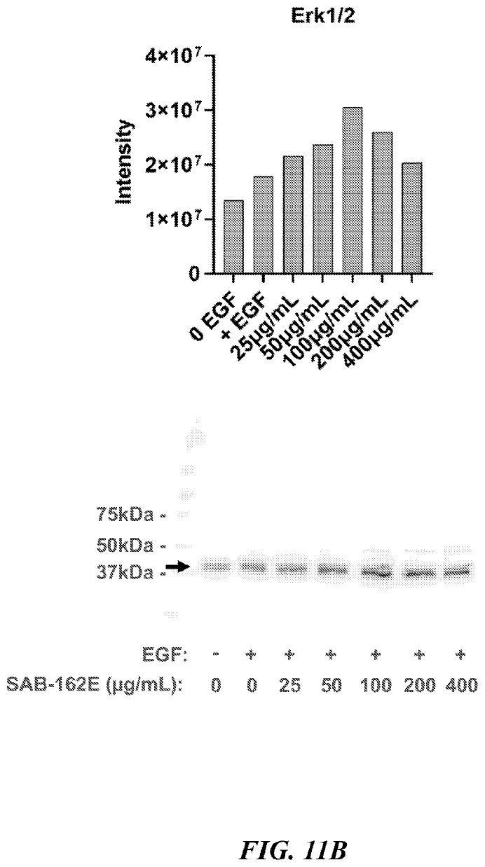

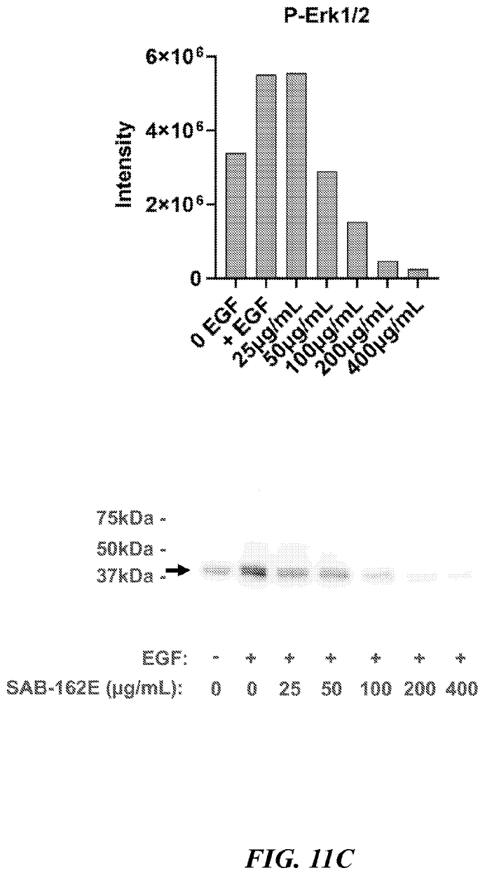

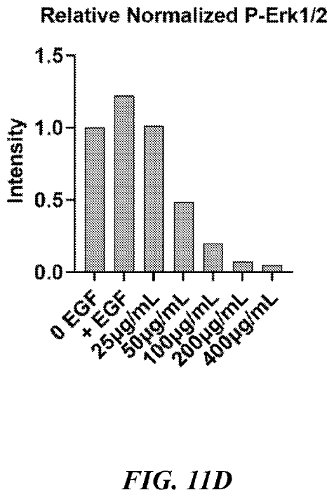

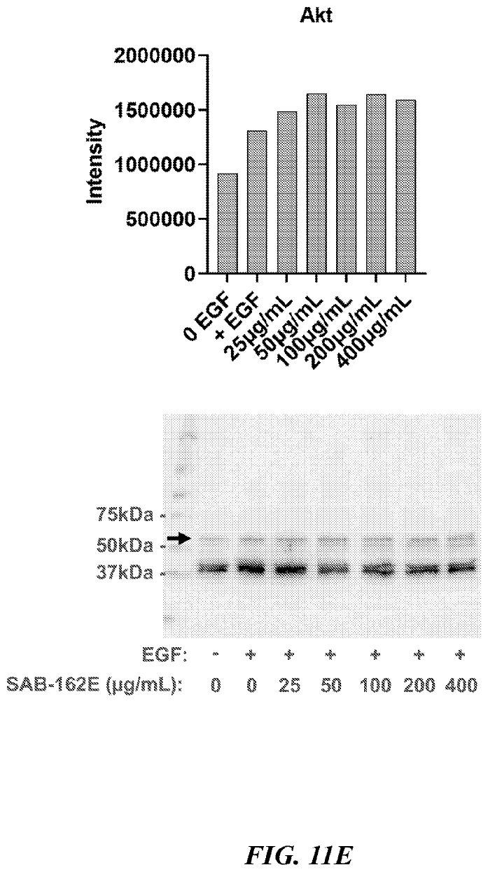

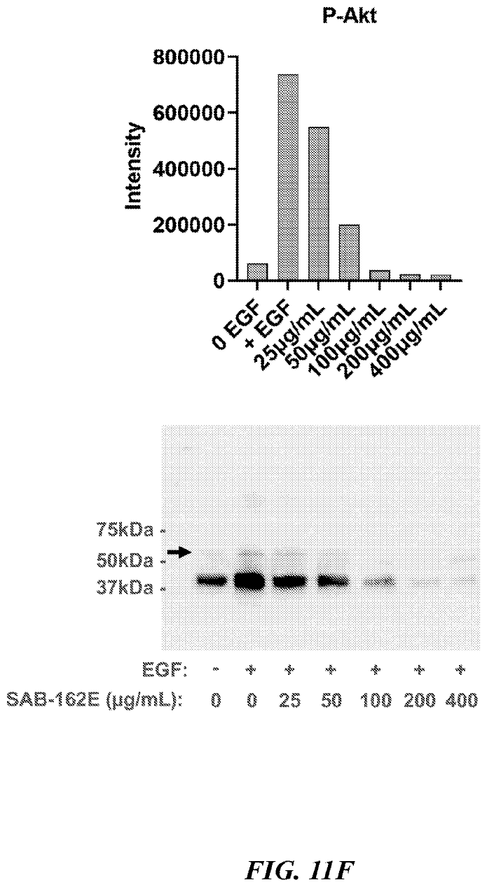

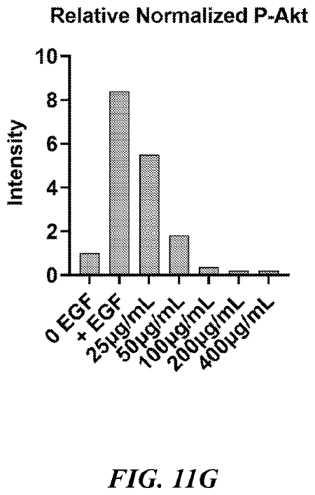

Binding of EGF to EGFR causes conformational changes in the extracellular domain of EGFR which facilitate homodimerization with another EGFR molecule. Upon dimerization, EGFR is auto-phosphorylated on tyrosine residues located in the carboxy terminus. Through this activation, EGFR mediates the PI3K/Akt/PTEN/mTOR and the RAS/RAF/MEK/ERK signaling pathways which control growth, cell survival, proliferation and apoptosis. Western immunoblot analysis was performed to determine whether SAB-162E blockade of EGF binding to the EGFR alters downstream cell signaling. For these assays, NSCLC H292 cells were chosen because they do not contain mutations in downstream signaling proteins. For example, a NSCLC cell line containing a constitutively active Ras mutation would convolute EGFR mediated signaling. H292 cells were serum starved, incubated with increasing amounts of SAB-162 and stimulated with the addition of EGF. For controls, cells were assayed in the absence of SAB-162E with and without EGF. Cell lysates were examined by western immunoblot analysis by probing with anti-Akt p44/42 MAPK (Erk1/2) antibody, anti-phospho-p44/42 MAPK (Erk1/2) (Thr202/Tyr204) antibody, anti-Akt antibody and phospho-Akt (Ser473) antibody (FIG. 11A). After developing the western blot, the intensity of the bands was measured by densitometry. The lowest amount of Erk1/2 was in the unstimulated cells without the addition of antibody. With the addition of EGF, levels of Erk1/2 slightly increased (FIG. 11B). For phosphorylated Erk1/2, the amount of phosphorylated protein decreased with increasing levels of SAB-162E (FIG. 11C). This finding is more evident after normalization of phosphorylated Erk to total Erk (FIG. 11D). This result indicated that SAB-162E is blocking EGR binding to EGFR causing a decrease in the phosphorylation status of Erk1/2. Upon the addition of EGF without SAB-162E, levels of Akt increased. Yet, levels of Akt were relatively consistent regardless of SAB-162E concentration (FIG. 11E). Lysates derived from cells incubated with 100, 200 and 400 μg/mL of SAB-162E had levels of Akt phosphorylation lower than the amount without addition of EGF (FIG. 11F), which is more distinct after normalization of phosphorylated Akt to total protein levels (FIG. 11G). SAB-162E indirectly decreases phosphorylation of Akt by inhibiting binding of EGF to the EGFR. This data suggests that SAB-162E would decrease the growth of NSCLC cells and be an effective therapy for NSCLC patients.

### ****

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Budapest Treaty Deposit

Immunoglobulins described in this application were deposited with the American Type Culture Collection (ATCC®), located at 10801 University Blvd., Manassas, VA 20110, USA. The deposits were made under the terms of the Budapest Treaty on the International Recognition of the Deposit of Microorganisms for the Purposes of Patent Procedure on Nov. 2, 2021. The ATCC accession number for the aforementioned Budapest Treaty deposit is Deposit No. PTA-127158.

1. An ungulate-derived polyclonal human immunoglobulin composition, comprising a population of human immunoglobulins, wherein the population of human immunoglobulins specifically binds Epidermal Growth Factor Receptor (EGFR).

2. The composition of claim 1, wherein the composition is produced by immunizing a transgenic ungulate with an antigenic fragment of EGFR.

3. The composition of claim 2, wherein the antigenic fragment of EGFR is an EGFR extracellular domain.

4. The composition of claim 3, wherein the antigenic fragment comprises, consists of, or consists essentially of an EGFR sequence according to SEQ ID NO: 16, amino acids 25-638 of an EGFR sequence according to SEQ ID NO: 15, or variants thereof.

5. The composition of claim 3, wherein the antigenic fragment shares at least 80% identity to SEQ ID NO: 16, SEQ ID NO: 15, or fragments thereof.

6.-15. (canceled)

16. The composition of claim 1, wherein, the population of human immunoglobulin composition is substantially similar to ATCC Deposit No. PTA-127158 or wherein population of human immunoglobulins has an avidity for EGFR at least 50% as great as that of ATCC Deposit No. PTA-127158.

17. A method of making polyclonal human immunoglobulin specific for Epidermal Growth Factor Receptor (EGFR), comprising administering an antigenic fragment of EGFR, or a polynucleotide encoding the antigenic fragment, to a transgenic ungulate, wherein the transgenic ungulate comprises a genome comprising a human immunoglobulin locus or an artificial chromosome comprising a human immunoglobulin locus, and wherein the transgenic ungulate produces a population of human immunoglobulins that specifically binds EGFR.

18. The method of claim 17 comprising administering the antigenic fragment or polynucleotide encoding the antigenic fragment 3, 4, 5, or more times.

19. The method of claim 17, comprising collecting serum or plasma from the transgenic ungulate.

20. The method of claim 19, wherein the serum or plasma comprises the population of human immunoglobulins.

21. The method of claim 17, wherein the antigenic fragment of EGFR is an EGFR extracellular domain.

22. The method of claim 21, wherein the antigenic fragment comprises an EGFR sequence according to SEQ ID NO: 16, amino acids 25-638 of an EGFR sequence according to SEQ ID NO: 15, or variants thereof.

23. The method of claim 22, the antigenic fragment shares at least 80% identity to SEQ ID NO: 16, SEQ ID NO: 15, or fragments thereof.

24.-35. (canceled)

36. The method of claim 17, comprising:
a) administering the polynucleotide encoding the antigenic fragment of EGFR;
b) administering the polynucleotide encoding the encoding the antigenic fragment of EGFR, three to four weeks later;
c) administering the antigenic fragment of EGFR, four weeks later
d) administering the antigenic fragment of EGFR, four weeks later, and
e) administering the antigenic fragment of EGFR, four weeks later.

37. The method of claim 17, comprising purifying the human immunoglobulin.

38. A pharmaceutical composition, comprising the composition of claim 1 and optionally one or more pharmaceutically acceptable excipients.

39. A method of treating or preventing cancer in a subject in need thereof, comprising administering an effective amount of the composition of claim 1 or the pharmaceutical composition of claim 38 to the subject.


SEQUENCE LISTING (16 sequences)

SEQ ID NO: 1 - DNA, 6802 bp, Homo sapiens
cagcagggtcacaagggcaggccgggtccttgtggagagcacatttagtgggagggacatgatttcccttcaaagtgcccattctggacgcttcccgttccatgctggacgcttcctcttccacgctggatgcttcctgttccacactggatgcttcctgttccacgctggatgtttcctgttacactctggatgcttcctgttccacactggatgcttcctgttccatcctggatgcttcctgttccatgctggacatttcctgttccactctggatgctccctgttccatgctggatgcttcctgttccatgctggatgcttcctgttccatgctggacatttcctgttccactctgcatgcttcctgttccactctggatgcttcctgttccatggtggacgtttcctgttccactctgcatgcttcctgttccatgctggatgcttccttttccattctggatgcttcctgttccatgttggatgtttcttgttccactctggatgcttcctattccattctggatgcttcctgttccatgctggacatttcctgttccatgctggatgctttctgttacatcctggatgcttcctgttccatgctggatgttttttgtttgactctggatgcttccagttccattctggatgcttcctgttccatgctggatgcttccttttccattccgcacaattcctattccattctggacacttcctgtgcgacacctccttgggttttctgtctgcccagtccctctatcctcatcccgttccctgctacctcccacctccacaatcgtccttgcccagctcctccctctctctagagcttcggcctggcaaggtccctcctgatctcagtccaggctcccccagcacaggtaggagcctagcacctgcccttggacctccccaccctgcatggtgccagcatcccccggtccccagggaggccccatttctctctctgcttgtagtccagtggccctggagtcccactgcaactcgggtgtgcccctgacctctgaggaagttaagtgtcctgtccctagccaggctatcccgtctgctcagccccagggccctgcccccaaccccttcccctcacctgcaccacaggctctggccaactctgcccaggccctgaatgggcccctctggctcccctctgctgctacactgccctgcaccacctccactcagcttcagtgtgttcatccacctgtcccaagtcccctcggcccccaggagcacagctggtggccctggctcctggcagcccatcttgttccttctggagcaccagcctcagaggccttcctgtgcagggtccacttggccagccctgggaccctcctggtctcaagcacacacgttctccctgcagccagacctgcccctgcctgtgagctcagccctgagccttggaatgccttcccatctccatcccagctcgcctttgccagctgctcagcaggatgaactcacactcccctccctgcaccatgagtcagagccagctggagacacgcccaggccaaagcagccaccagggcctagtgggggccagaagcttcagatgagaggcccaggtattgagaggctgagatcacgggcagaatggtcataatcgctgccagtatcagtccagccccagggactcagagacagagaaaagagcagcacacaaggtctgggctccccaccttctcccgtgagtacgggggagtatgggggcagccaccacccccatccccacacacccatgaggcagcctcggctgtgtctggactccccctcgccctctgacccagaaaccaccagaagaaaagggaacttcaggaagtaagtggtgccgccggtttcaatcctgttcttagtctttgcagcgtggagttcacacacctggggacctgggggccgagctgtgatttcctaggaagacaaatagcggctgacggcaggggcggggctgcccacatgtacctcgccagaacaggaagggctgagacccccacctcggtgagtggggtcagcacagggcaggggcacaggctcgggaggaggacagagcctgggggcagccgtgggcgctcctggacctgagatgctgaacaggctccaagaggctggggagacatggggtcgaggccggccccacatggaggcccaagcggagccagcacgggggaggtgggcagccttcaggcaccgatgcccacccagtgcgagacgacggggaccgtgggcaggggcttccaagccaacagggcaggacacaccagaggctgactgaggcctccaggacgaccgggctgggagcacgaggaacatgactggatgcggcagagccggccgtggggtgatgccaggatgggcacgaccgacctgagctcaggaggcagcagagcgagggaggaggagaggccccaggtgaacggaggggcttgtccaggccggcagcatcaccggagcccagggcagggtcagcagtgctggccgtggggccctcctctcagccaggaccaaggacagcaggtgagccgggagcagagcagggagggtgagtgtggcagcaggacaggagggtggaagccaaggagcccagaggcagaggcagggacaggggaggcacaggggctgggctcagagccagctgatggggttggggcacctgctggcggggagcagggctgtggtcagcagtggagaggaggggagagctgtgctgagtgcacgggcgggagaagggaagagtccagggaggcccagaaaggcccagagtgcagcaggcctggggcgaggggaagggctgaggctccgtgcgttcagggaactgacccagcagagcagaggccactgaggagctgaggttccagagaggcttccagagcaggagcagtgcagggacaggaggatccgggagctcattcaggaggggcacatgggcaagggcaaggggctctgttggggagacctgactggacactggggctgctccacagcatagggaacacgccaagtgctgcaaaatcaaaaatgagggcagaaaaacagcccaaacctggacagagggtgccaggacaggcaggggggcaacagtgacctgagtgacattgctgcccgggttgagggagggcagagtgagcagggagcaggcattggagctcagggaccaggaccaagcagccacaggtgagcagggcaggtgggggcagaaggagcagggggcacctcctggagctcaggggaccagggcagagcagcctcaggtgagcaggggctggtgggcggcaggatgagcagggggaagaccctggagctcaggggaccagggcagagcatcagaaggtgagcatggctagtgggagatgggcaagcagggggcagcccctggagctcaggggaccaggacagagcatcaggaggtgagcatggctagtgggaggtgggcgagcagggggcagcccctggaactcaggggaccagggcagagcagccgcaggtgagcacgggctggtgggaggcgggaggaacagggggcagctcctggacttcaggggaccagggagggcatctgaaggtgaacaggggccagtggggggcaggatgagcagggggaagctcctggagctcagggagccaaggcagagcagccgcaggtcagcaggggcaggtgggaagcatggggagcaggtgggcagcccctggagctcagagagccagggcagatcatccacaggagagcaggggctggtaggaagcaggaggagcaagtgggcagcttttggagctcagagcaccagggcagaagagcctcaggtgagaaggggcaggtgggaggcagaataagcaggggacagcccctggacctcaggagaccagggcagagcatcacaacgtcagcatggctggtgggaggtgggcgagcagggggcagcccctggacctcagagagccagggcagatctgcaggtgagcaggggcaggtgggaggcaggaagagcaggaggcagctcctggagctcaggggatcagggcagagcagccacaggtgagcaggggcaggtaggaagcagaaagatcaggggtcagcccctggagctcaggggacaaggggagagcatcagaaggtgagcaggactgaggctcagcctcagggagccagggcagagcagctgcaggtgagcagggccggtgggaagcaggaggagcaggtgggcagcccctggagctcagagagccagggaagatcatccgcaggtgagcaggggctggtgggaagcaggaggagcaaggggcagctcctggagctcaggggaccagggcagagcagtcgcaggtgaacaggggcaggtggggggcaggaggagcaaggagcagctcctggagctcaggggaccagggcagagcagtcgcaggtgaacaggggcaggaggagcaaggggcagctcctggagctcaggggaccagggcagagcagccgcaggtgagcaggtgcaggtggggggcaggaggagcagggggcagctcctggagctcaggggaccagggcagagcagccgcaggtgagcaggggcaggtggggtgcaggaggagcagggggcaggcactggagctcaggggaccagggcagagcagtcgcaggtgaacaggggcaggtggggggcaggagtagcaaggggcagctcctggagctcaggggaccagggcagagcagtcgcaggtgaacaggggcaggtggggggcaggaggagcagggggcagctcctggagctcaggggaccagggcagagcagccgcaggtgagcaggtgcaggtggggggcaggaggagcaggggtcaggcactggagctcaggggaccagggcagagcagccgcaggtgagcaggggcaggtggggggcaggaggagcagggggcaggcactggagctcaggggaccagggcagagcagccgcaggtgagcaggggcaggtggggggcaggaggagcagggggcaggcactggagctcaggggaccagggcagagcagccgcaggtgagcaggggcaggtggggggcaggaggagcagggggcaggcactggagctcaggggaccagggcagagcagccgcaggtcagcagggccggtgggaggcaggacgagcaggggacaggcactagagctcagggcaaggcagccacaggtgagcagggctggtgggaggcatcactcagctcctagactttggcaggagctgggtagttgctggcaacagacagctgagggctggtgaaagtgcagtgcagcctcctggtgccgggaagggagtgtgagtccatcccactgagcagttggcaagggcgagctgggatggagaagggaaggcgttccagggctcagggctgagctctcaggcaggggcaggtgtggctgcagggggaacgtgtgcttgagaccaggagggtcccacggctggtcccagcggaccctgggcaggaaggcctctgaggctggcgccccagaaggagcaagatgggctgccaggagccagggccaccagcacaatgaagctgagtggaggtggtgcagggcagtgtagcagcagagggcagccagaggggcccattcagggcctgggcagagtcagccagagcctgtggtgcaggtgaggggaaggggtggtgagcggggccctggggctgagcagaggggatggcctggctgagggcagggcgcttagcctcctcagaggtcaggggcacaccccacctgcagtgggactccagggccactgggccagcggcagagagaaatggggcctccctgtggcctgggggtcctggcaccatgcagggtggggagggccaagggcaggtgcaaggctcctacctgtgctggggggcctgggttgagcccagcagggaccttgccgggggaagctctggagagagggaggaggtgggctggtggccgagaaggccaggccagggctgggagggtgacggtgtggtgactgagcctccagaagtaatgcaggacactgggaggcagggggcatccaggcactcagggccctgacctgggctgctgcacactggggctaaggggaaaggaggggagaggctgaggaggaggctccaggaggctattccaaggcagggggttccggggccctggggctgaagggcgccgaccctatgcagtgtctggcccctctgctgcacagaagaaaagggccttggagggcagagggcaggctatgaccagggccctgggcaagtcaggccaactcactaggggagggccacgctggggcggcagggtcaggggcttcagggggctcgggggacccacgagaagccatctgagaacagtgtccactggtcaagccaggcacccataaaaggctggagtggggccaatgggcatgagccgtccctgaggtggcaccgatggccagagctgaggccaagctagaggccctggactgtgctgactcccggcagacacagagcgctgacctggctgccgagccccgcctcctaggctgcaggggtgcctgcagaagggcaccacagggccaccggtcctgcaagctttctggggcaggccgggcctgaccttggctttggggcagggggtgggctaaggtgacgcaggtggcgccagccaggcgcacacccaatgcccgtgagcccagacactggacgctgaacctcgcggacagttaagaacccaggggcctctgcgccctgggcccagctctgtcccacaccgcggtcacatggcaccacctctcttgcag

SEQ ID NO: 2 - DNA, 9483 bp, Bos taurus
gatcaaacactgtcaaaccaacacaagagagccccagtaccagatgtgaggctccccccagaccccagtcacagtgcagcctgtagagggaacaggcccagtcgggagaccaggaccgagaaagtgacccctgtcagtcagtccagctactcagtcatgtccgactctttgcaaccccacagactgcagcacgccaggcctccctgcccatcaccaactcccggagcttgctcaaactcaagtccattgagtcggcgatgccatccagccatctcatcctctgtcgtccccttctcctcctgcccccaatccctcccagcatcagagtcttttccaatgagtcaactcttcgcatgaggtggccaaagtattggagtttcagcttcaacgtcagtccttccaatgaacacccaggactgatctcctttacaatggactgattggatctcattgtagtccaagggattctcaagagtcttttccaacagcacagttcaaaagcattgattttttactcagctttctttatggtccaactctcacatccatacatgactactggaaaaaccatagctttgactagacggacctttgtcagcaaagtaatatctctgctttctaatgtgctgtctacgttggtcatagcttttcttccaaggaacaagtgtcttttaatttcatggctgcagtcaccatctggagtgattttggagcccaagaaaatagtctgtcactgtttccattgtttcctcgtctattttgccatgaatgccatgatcttcatttttcgaatgttgagttttaagccagctttttcactctcctctttcactttcatcaagagtctctctagttcctcttcattttctgccatgagagtggtgtcatctgcatatctgaggttattgctatttctcctggtaatcttgattccagcttgtgcttcctccatccaggtatgtcacatgatgtactctgcatagaagttaaataagcagggtcacaaaatacaatcttgatgttctccttttccaaattttaaccagtccgttgttccttatctggttctgttacttcttgacctgcatataggtttctcaggaggcagttaatgtggtctggtattcccctctctttaagaatgttccagtctgttgtgatccacccattcaaaggctttagtgtagtcaataaagcagaagcagtaaaatgctgccactttctaggtggagctcacatatggataccacacccaaaccttgaccaagccagaacctcatgacaatcatgtgcagtgctataaatgatctcagctgttacacagtcactatttacaaaatgtggggcaagagagcctgaacatgccatcttggccaattttctctctcctcatgggttgtttagctttttttctttttttttttttttaattaagctgtatgagttgcttgcacattttggaagtttattctttagtggtcacgtgatttgcaaatatcttctctaattctgcaggtagtcttgttttgttgatggttttccatggtgtacaaaagccttttagcttaattaggtcccatttgtttatttttctttttatttctattactctaggagatggatggagaaggaaatggcagcccactccagtgttcttgcctggagaatcccagggacgggggagcctggtgggctgccgtctatggggttgcactgagtcggacatgactgaagtgatatagcattagcattaggagataaatacaaaaagacaccaccttgatttatgtcaaagagtgttctgcccatgtttccctctgttttatagcctcccatttcacacttttgtctttaatccattttgagtttatttttgcccatggcactagagaatttataatttcatttattaacaggtagcaatccagtttttccagaacttattcaagagactgtcttttctccatcttacactcttgcctcctgggtcatacatgaattgatcataggtgggtgggattgttcctgggctgtctatcctgtctcactgatcactgatctataattgtggttttgtgccagcaccatagtgttttgatggccgtagctttgcagtatgctctgaagtctgggagcctgatgcctccagctccacttttgtttcttgagattgctttgactatttggggtctttatgtctcccaacaaattttcaaaagttttgttctgggtctgtggaaattgccattggtcatttgataggtgttacactgaacctgtagattgctttcggtagaagtgtcattggcacaatattgattcttctgatttaagagtatcttttctaaatagacatttctccaaaaaagacatacagatggctaacaaacacatgaaaagatgctcaacatcactcattatcagagaaatgcaaatcaaaaccactatgaggtaccatttcacaccagtcagaatggctgcgatccaaaagtctacaagcaataaatgctggagagggtgtggagaaaagggaaccctcttacactgttggtgggaatgcaaactagtacagccactatggagaacagtgtggagattccttaaaaaactggaaatagaactgccttatgatccagcaaccccactgctgggcatacacactgaggaaaccagaagggaaagagacacgtgtaccccaatgttcatcgcagcactgtttataatagccaagacatggaagcaacctagatgtccatcagcagatgaatggataagaaagctgtggtacatatacacaatggagtattactcagccattaaaaagaatacatttgaatcagttctaatgaggtggatgaaactggagcctattatacagagtgaagtaagccagaaggaaaaacataaatacagtatactaacgcatatatatggaatttagaaagatggtaacaataacccggtgtacgagacagcaaaagagacactgatgtatagaacagtcttatggactctgtgggagagggagagggtgggaagatttgggagaatggcaatgaaacatgtaaaatatcatgtaggaaacgagttgccagtccaggttcgatgcacgatgctggatgcttggggctggtgcactgggacggcccagagggatggtatggggagggaggagggaggagggttcgggatggggaacacatgtatacctgtggtggattcattttgatatttggcaaaactaatacaattatgtaaagtttaaaaataaaataaaattggaagaataaaaaaaaaaaaagagtatcttttcatctgtttgtgtcaccttaatttctttcatcagtgtcttagagctttcagagcagaggaattttgcctcctagggaaggtttattcctaggtattttattctttttgatgggatggtaagtgggattctttccttaaattctctttatggtatttcattgctgccatacagaatgcaacagatttttgtgtattaattttgtatccataaattttatccaattcattcctgagctctagggttttctgtaccttctttagcattttgcatgtgttaaatcatgtcacctgcaaacagcaggtgtttcttacttggcttctggattccctgtgtccctttctctcccctgattcttacacttaggccttcactgtgctgcactgtgggcccttgctggttacctactatatacgcagtcgtgtggatgtattagcaccaaactcctaatttatccctgggatgtggtgggtcaaccaggcgcagtgcatgaggaagaggaacaccgtgacggctaggattcccactcctcccctgggtgcaagggcgccgcttaaaccaccccaccaacccacaagacatacctcagtcacatctggactcccccaggccctctgagacagaaaacactcagaagaaaagggaacttcaggaagcaagtcgcgtcaccaggtttcattcctgttcttagtcttcacagcactgggggaagggccctcacacctcctgggactggtgaccaagtcccagggagcagggctgcagacacagaacacagggaacttcaggaggcagatggagtcaccaggtttcattcctgttcttagtcttcacagcactgggggaagggccctcacgcctcctgggactggtgaccaagtcttagggagcagggctgcagaaacagcacgcagatcccaggagccacatgtgtctgtgggccagagcagggaaggggcccggagccccaggctgtggggcagccccctccatggcccccaccatgggtgagtggggaccccgaggatgagcgcacagggacaggaatgagagccctggagggagaccggcctcgggacgcccagaggggctggagcagccgagtgggcccaggggaggcagctgcccctggtcacagtgcaggacgagcgtgtggacagaacccagacagggtgcaccacaccaagagcactgctgcccagggctggagagctcagggggctgtgggccgtggaggagcaggatgggcttgggggcgggagggaagggatgtgcatgaccaggggctctggaggccggggctgggcagtcatgaggaccagccaggacggaaagagggtctgacctgaggacgcagggaagggagaggaccccagagcagtgaagtcttctggacaggccagtggagccagcgtcggtcccagggcagggccacatgcagggactgtgggtcccgactgggctggggccgagggcagcggcagacttgggggcaaggctgcagggccagccgggttctatgtccacagcaggcaccacggggctgaacccacagccccccaggacacagggggctgaccagagctcagggcctgaggaccagcagtgggagccagagagaggcaggtgacccgaaaactggaggcagcaaggggccagaggagggtgtggatggagggatagagaagagcagagagcaaaggcaggacctctgccaggaggggcccgggtggacagagctcctccaggggagagttgcctggaggccagtgggcacctggcaagtccagggatgagagctgaacccaggaaataaaggagtgaggagtggagatgggcaggggtgagggcttccagaggggccagaggggcaagaaaggctgagggggcggcccaaggacagggggcagcaaggctgcaggtgagcaggggctgggagggcaggggggcccctagagatctgggggagcaggcacagcttgtgggtgaaaaggggttaggagggcagggggcccctggtgtttgggggagcagatagagcttttgtaggtgagcagggagctgggagggcgggggagagggtggcctggtgctttggggagcaggtacagcctgtgagggtgagcaggggctgggagggcagggggtgttcctggtgtttggggtagaaggcacagcttatgggtgagcaggggctgggagggcagggggagcccccggtgctttgggggagcaggtactggttatgagggtgagcaagggctgggaggacggggggagcccccagtgctttgggggaacaggtacaggttatgagggttgagcagggggctgagagtaaggaggtgagcctagtgctttgggggagcatgtacagcctgtgaaggtgagcaagggctaggaggacagtgggatcccctggtgctttaggggagcaggtacagcttgtgagggtgagcaagggactgggaggacaaggggtgcccctggtgtttgggggagcaggtactagttatgagggtgagcaggggctgggaggactgggggagcccctagtgctttgggggagcaggtacaggttatgagggtgagcaggggctgggaggacagggggtgttcctggtgtttggggtagaaggcacagcttatgggtgagcaggggctgggaggacaggggggcccctggagatatggggaagcaggtacagcttgttggtgagcaggggctgggaggacagggggagcccctggtgctttgggggaacaggtacaggttatgagggtgagcagggggctgagagtaaggaggtgagcctagtgctttgggggagcatgtacagcctgtgaaggtgagcaagggctaggaggacagtgggatcccctggtgctttgggggagcaggtacaggttatgagggtgagcaaggggctgggaggacaaggggtgcccctggtgctttgggggaacaagtacagcctgtgacggtgagcagggagctgggagggcagggggtgcctctggtgctttgggggagcaggtacagcttgtgagtgtgagcaagggctgggagaactgggggtgtccctggtatttgggggagcaggtactggttatgagggtgagcaggggctgggaggactgggggagcccccagtgctttgggggagcaggtacaagttatgagggtgagcagggggcttagagtaagggggggtgcacctggtgctttggggagcgttacagcctctgagggtgagcatgggctgggaggacagtgggatcccctggtgctttgggggagcaggtacagcctgtgagggtgagcaggggctgggaggacagggggagcccttggtgtttgggggagcgtgtacagcctgtgagggtgagcaggggctgggaggaaagtgggagcccctggtgtttgggggagcaggtacagcctgtgagggtgagcaggggctgggaggaaagtgggagcccctggtgtttgggggagcgtgtacagcctgtgagggtgagcaggggctgggaggaaagtaggagcccctggtgtttgggggagcgtgtacagcctgtgagggtgagcacgggctgggagggcagggggagcccctggtgctttgggggagcaggtacaggttatgagggtgagcaaggggctgggagggcagggggagcccctggtgtttgggggagcatgtacagcctgtgagggtgagcaggggctgggagggcagggggcgcccctggtgttttggggagcggtacagcttgagagggtgaacaggggctgggaggacagggggagcccctgatgctttgggggagcaggtacagcttgtgagtgtgagcaagggctgggaaaactgggggtgtccctggtgtttgggggagcaggtactggttatgagggtgagcaggggctgggaggactgggggagcccgtggtgctttgggggagcaggtacaggttatgagggtgagcagggggctgagagaaaggggggtgtgcctggtgctttggggagcattacaatctgtgagggtgagcaggggctgggagggcagggggagcccctggtgctttgggagaacaggtacagcctgtgagggtgagcaggggctgggaggacagggggagcccctggtgtttgggggagtgtgtacagcctgtgagggtgagcaggagctgggagggcagggggagcccctggtgctttgggggagcaggtacaggttatgagggtgagcaggggctgggaggactgggggtgtgcctgatgctttgggggagcaggtacagcttgtgagggtgagcagggggccgggaaagcagggtgtatctcaaaaactttaggggatcaggtagtgcttctggggatgagcagaggtctgggaggacagggggtgcccttggagttggagagcaggtacagtctgtgaggtgagtagagggctgggcaggcacagggagcccctggtgctttgggggaacaggtgcaggtcgtgagggtgagcaggggctgggaggacagggagtgtccctggtgtttggggtagaaggcacagcttatgggtgagcagggcctgggaggacagggggagcccctgttgctttgggggagcaggtacagcttgtgagggtgagctgggtctgggaagacagggggagcccctggtgctttgggggagcaggtacagcttgtgagggtgagctgggtctgggaagacagggggagcccctggtgtttgggagagcagatacagcctgtgagggttagcaggggctgggaggacagggggtgcccttggagttggagagcaggtacagtctgtgaggtgagtagagggctgggtgggcagggggagcccctggtgctttgggggaacaggtaaaggttatgagggtgagcagggggctgagagtaagggcgtgtgcctgttgctttggggaagcatgtacagcctgtgagggtgagcaggagctgggagggcaggggggagcccctggtgctttgggggagcaggtacagcctgtgagggtatccagggggctgggaggacatggggtgcccctggagatttttggaagcagtaatagcttgtgggtgagcaggaagctgcgaggacaggaggtgcccctgttgctttgggtgaacaggtacaacttgggtgagcaggggctgggaggacagggagtgcccttggagatttgggggagcaggtgcagcttctggggataagcagggggctgggagttcagggtattaggggagcagacaaagatcgcgcagctgagcaggcgctggaatgtcacgcccctgccgacgacctcagttgaccatgtgtgtgctgagcacatcggtacgaaagggtccgagggtgcaaggggccatttgtgctgtgggccagatgcagggacacagggcagtgtgagccctgcagagaagatggggcccaagagcgcagctgtccagagctgagtccagagggctgagaccagtggtgtgggtgctggacgagggtgaagggctggcgtgagggagcaagggcacagggctgggagagctcaggccacagctggtgaagcagagggctgagacaaggggtgtaggccacccctcaagacaatgggggtgctgtttggtggagggtctgcatgaagaacaaagcaaggaggagcgaagacccgagaggaagggctctggggctgcagggaaggggcgccctgagggcaccgggtgggctgcacttctgggccgagggtctgcctgtcccagcagcctgtgtggctcaggagccaaagtggggccagacctgggacgctgtgcttgatagggcggtgcaaggggccaggagctacagggcgcccactggcagtcagtcgggctgcaggcccagggagctgaccaggctgggggagcaggggccatgggaagggccaggcgcccacaggagggaaggggccctgggctctgatccaggaggccacactcaccgacccaagctgtgtccagacaccccaactgcagtacagagagccgggtggccaccatgccggccggtcatcagaccctggaagcaggtggtggctgggctcggaggtgccccaggcctgggcacctgaggtcctgctggaccccgcattcacccagcctcctctctcacag

SEQ ID NO: 3 - DNA, 10298 bp, Equus ferus
  misc_feature (6918)..(8037): n is any nucleotide
agatatcccccaagtcatgtcacaatactaccatgttcaaagtacatgaatgctttgaaacagactggtctcagtggctgggtgttccttgggtggctcctaatggaactcagtggttgtgtggaacaaacctctagtcttggctcctgcctggatggctgggacactgcaccctggacttcctttggaggcagggaaggagtcgccctgagctaacagctggtctcaatgttcctcccagtaaggccagatggactccatcagtgttcactggtatgaccaccagcggctatatttgctgcttccttgggctacaagatgtcatatcacatagaggctctaactaaatttactcaaactgccctcaatgaaagtcaggtaggaatacccttattaaatactcaaatgtctttagtggggagggctgtctccccatcagaatggccttagatagtctaacagcatcccaaggtgggacatgtgcactcattcaaactgaacgctgcgtttttagacctgatgagtcttccaatgtgtcctctctgctaaagcacttggaaaagcaggtaaatgccttaagcgatcctgcatccaggcttgatttgcgtggttggctcccttcaggtgtggctgccctcttgaaatctgaattgcaattcctgtgtctgttactccttggaattcttctattaatcataatatgcaaactaattactctttcctttactcagtgttgtaagactggcatgcaggctagaataatggttgctcagccacttgaggtgattgattgatcttacaaccctggatgaatttccttctcataagactatgcctaagaactcatttttcaaataatcctttcaagttttgaattatcagcactgttagtgtagctgttctataattaggcacatgcagcatcatgagtcagatcaaagcataggtacaacgtttgtgtgacaatctaaagttactgaaaacttacataacctatggaatgcttcccctgtaattgtatacctctatgctcatccactatatccaactatttcctgccattgggcataactgagcacgtcaatgagataaaagcccagcactgatggccatcatggaccagggcaggcaacaatcaccatagcaccaagggacaatatttagatcatcagtgctttctataaaaacattatcgatcaaaggtgggaaaataatgaatcaatgaaagcaataagaaaagttaaaatgttgaggtatatgaggaagccatttggtttagactaattaggcctcatctagtttttctggaaagtcctgatgtgcctgtcgaatatgcattgtacatctgctgcatttacaatgtcccaaagcaagaatgatgcccttgaaattatcctgcccccttttggcatttctttatagatgagcacttcttcccagaatgcaaggattcgttactgacctactgtgtcatcttgtgatcactcacccgcaatactagcaaagcatcttgtgactgtagtaaaagagatactcctgtcatatgtgatgtacgtcccttgttccaagacggtatataaccacgctgcacacccggcttcttcacaacacttccttccttggtgaaggttattgtcccgggctatgtagtcctcaaattggctcaaataataaactcaccccaattttgattaacagattgattatagtttattgcctcaacatttcctgaccagaaattacatagatgaacgtgcttcttatgtaacggcagcgatgctcacagcctgcggagcacatgactgagggaggctgccttccgtgggagagatggcaaaactgcgtgggcgccatggagaggaggccgggccagagccgccgggagtgtgcagtggattatcatgaattcatcccggagggaccagagggcactcccttcaccaaggctgctgctgacggcgttttctgggtgtccacagcaacattcccaggaccacaggctcctccatcacctggtcctgccccatgcagaggttcacttccctcaccagaccccagggtacccatgcgactgactccatggggacagggccactgagggaccactgctgggtctctgagagcaggacctaaggagacagggctcctcctgggagtcaggcccgggcaccactcagcgtgggcaatccccatggaaagatcacagagacagagggaacacgggcgcccgcccagcccaggcgccagactccaggtggagccttccagcccctcgctgatgcgagtggagcagagaggagcgcccccgcccacagaagccggcactgcctgcaggccaccaacgcagaagcaaatcagcagaagagctgcagaggccactctctgtggggcggtccacagcacacccatgaaccgccggacacgatatgggacccgcacgtgggcgctgctctgagaaacctcaactctagaaagtaagcacgaggaccagagagcaggggaggcagagggacccagagtagaccgttgggggatgtggagtggctctggggacacccgaggccaaagggtgctgcaaatagcaacaggtgggagggccgactcccctcgtacatctcaggtcacctcctagaaggttcgctttggactttgctcttggcccacctaagcggccacagcgtacgcccctgcccaccaaccattggaaggagccaggcctgccttcacgtccagtcacctacctggcccccggcaattccagggtggggaccatggtgccgcccagggatccccgatccaaggcctaacgtgcaagagacgggatggcccaggtttgtacacggggccttgggtgtgccgaggcactgcaagtcatgagacactgagggtccgtgtccccccacagacaaaaagctgggtcctggcccacacacgggaggacaggaacagcatgctgtctctcagacagaggagacctttgggccccagtgaccaccgtggactctgtccttgtacatagacttctttctgtccccagaggacagctgtggatgggagaatatcttccctgggaccctgggtgctaccacccttcagtcagagatccagccatggatggagccagagagggatggagggaagaggcagggacccagaggaagacggctttgtacttagggggctggcctggcaggaggacaggatgaggccctgggctgagctgcgggctgtgagcaggacagcctgtgtccacaatggacgctgactagggcagggggaggtgtcctctgggctgtgggtaccagagggctcaggtgagaggcctgggtgccctggcctttggtggggccatcgggcataatccttgtgggagggagagagcaaagagaggcatgtgagcccggacttcctgcactctccccagagtcctgggcgccagaccccctccactcccacacacccacgtgtggcagcctctgcctcacgtctgtactccccccggctctctaagacagaaaccacccagaagaaaagggaacttcagggagcaagcggtgcccctgctttcagtcccgttcttagtctttgcagggtcgtggagagtgggttcccacctccgggaaccagctaccacctccgggaaccagctaccaactcctaggaagcagggcagagggcaacaagcccggggccgcgaggagccacatgtgcccggggccagagcaggggcgtggggggagctcctcggaagccctggcttccaccacctccacagcccccacccttggtgagtggggtccccacggagcagggggaagggagtggagtcagggcagtggaaacggttgcagagctacggggagcccaggcacccagggacgactccgaggggctcaagcagctgagccagcacgggggagggcaggctgcccacgagccacggtgccaaccggacgcaggcaccaggcagccatgggcagggcacgcccacgccaacagagctggacacaccagaggcccatgaaagttaaccttgaggaaattgatgaccttgaggccagaagcctgagggctgcatttggccagctgtgctgagggcaggaccagccaggagctgagctgctgcagcacggacgccaggatgcacagatgggtttgagcacagagagggggctggctcagagcaggggaggaagaggacctccaggtggcctaagggtcctgtccacgccaatgagccagctcaagccccagggcagggtcagaggtggccgccatggtgtcctaggctgtagccaggaccgagggcagcaggtgagccaggtgcagagcccaaagggccggcggatgcacccatgtgctcagaactcagactgcacagggctgggcaggagctcagtggctacaggcagacgttgggagccaggcagtgagggtccagagcaggtgctggggttgcagtgttgggagctgggtggcaaggggagctgacccagcaatggagggagcacagtcccgaggggccttccagatgggcttctgagcgccgtgaggggcacagagacggtgaagcccagctgggccaggagctcctccaggaggagaccacggcaggcaggggctctgccaggggagagttagccagagagagggtgggcatctggtggctccaggaggcaaaggccagtgatgggaaaggaatgagcccaggatggagggtgaacctgggctgagggtgctgggagaggcaggaggggcaaaggcagccccgggcagacccagcagcctggggctgagcttagggacctgagctgtgtggaggggatcgtaggacaggagggacagagggctgctgtggacctggagttccagggacaacgggaagaacagctacggatgagcaggggaaggtgggagggcacaaaaggacaggagcctacaccaagagatgcatgggggcaggcagagcagctacaggtgagcagaggccagtgggagggctggagggaaagggggctgcccaggggctctggaagggggcaggtagagcagctacaggtgagcagaggaatgggagaccaggagggacaggtcacagcccaggagccccatgggtgtggagagcagagcttggcatgtaagtaggaagcaggaagggctgaaggggcaggggctacccagcagctccacaggagcaggcagagaagctacaggtgagcaggggccggtgggagggctgcaggggcagggggctgcccaggagctccggggagcaggcagagcagctacaggtgagcaggggccgatgggagggctgcaggggcagggggctgcccaggagctccggggagcaggcagagcagctacaggtgagcaggggccgatgggagggctgcagggacctgcccaggagctctggcggagtagtcagagcagctacaggtgagcaggggccagtgggagggctggaggggcagggtcctgcccaggagttccaggggagcaggcagagcagctataggtgagcagacgctggtgagaggtctggaggggcagggggctgcccaggagctctagcgggagcaggcagagaagctataggtgagcaggggccggtgggagggctgcaggggccggggcctgccgaggagctccggcggggtagtcagggcagctataggtgagcaggggccggtgggagggctggaggggcagtgggatacccaggagctccgccagggtagtcagagcagctacaggtgagcaggggcaagcaggagggctggaggggcaggaggctgcccaggagctccggcggggtagtcagagcagctacaggtgagcaggtgccagcgggagggctggaggggcagggggctgcccaggagctctgggaggagcaggctgagaagctgtagttgagcaggggccagtgggagggctggaggggcaggcggctgcccaggagctccgggggagcaagcagagcagctataggtgagcaggggcaggtgggggggctggaggggcagggcaatgcccaggagctccggcggggtagtcagagcagctacagtggagcaggtgccagcgggagggctggaggggcagggggctgcccaggagctccgggggagcaggcagagcagctacaggtgagcaggagcaagtgggagggctggaggggcagaggactgctcaggagctccgggggagcaggcagagcagctacaggtgagcaggagcaagtgggagggctggaggggcagaggactgctcaggagctccaggggagcaggcagaacagctactggtgagcaggggtcagcgggagcgctggaggggcagggggctgcccaggagctccgggggagcaggcagagcagctataggtgagcaggggcaggtgggagggctggaggggcagggcaatgcccaggagctccggcggggtagtcagagcagctacagtggagcaggtgccagcgggagggctggaggggcagggggctgcccaggagctccgggggagcaggcagagcagctacaggtgagcaggggccggtgggagggctgcaggggcagggtcctgcccaggagctccacaggagcagacagagaagctataggtaagcagggccggtgggagagctggaggggcagggtccggcccaggagttccgggggagcaggcagagcaggtacaggtgagcaggcgctggtgagaggtctggagcggcaggtggctgcccatgagctctggggggagcaggcagagcagctataggttagcagtggccagcaggagggctggaggggcagggagcagttcaaggagctccagcggaataatcagagcagctataggtgagcatgggccagcgggagggactgggacggggcagggagctgccaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttccagggaatcttctacagggagctagatagagaaagtttataggtaaagcaggtgccggtcgggagaagttgaaggggcagggtcctgccccaggaagttcccggggggagccaggcagagcaggttacaggtgagcaggcgattgtgagaagtctggaagcggcaggtgtcttcccatgagctcttggggggagcaggcagagcagctataggttagcagtggccagcaggaggggctggaggggcagggggcagccaaggagctccagcggggtagtcagagcagctataggtgagcaggggccagcgggagggctggaggggcaggggacttccaaggagctccatgggtgcagacagagcagaaactggtgagcaggggccggtgagagatctggagttgcagggggctgcccaggagctccgggggagcaggcagagcagctactggttgcaggggcaggtgggagggctggaggggcaggggcctgcccaggatctccagggagcaggcagagcagctataggtgagcaggggcaggtgagagggctggaggggcagcgggctgcccaggagctccaggggagcaggcagagcagctactggttgcagggcaggtgggagagctggaggggcagcgtcctgcccaggagctccacaggagcagacagagaagctataggtaagcaggggccggtgggagagctgggggggcagggtcctgcccaggagttccgggggagcaggcagagcaggtacaggtgagcaggtgctggtgagaggtctggagaggcagggggctgcccacgactttaggggatgaagggcactgggcctgagttaaacagagccctcaactggtgggacctgagggggtacagggagcagggacagctggaggctcccagggctcaggtcaggctggctgggcagggaggagagttgagctggttgagttgccaggtgcctgttgagttggatggaggggggcccctgggatggaaggagtctagggctgaaggggtcgggggcatccctgagaggaccagatcggatgcagttcttgggtccaggcacaccggccctgttctggggtttgtggggacatgggacaggagggatagacattcggactcagtgtggggtggcacagcgcagtgggctcagtgcatggaagagtaagagtccccttggggcctgagcccaggctaccagcatggggagaggccaggcaaggccctgcggctgagcagacaggactcaggggaggaggggacagggtgggcctggctgttgggggtggaggctgcaccctggcccacacgggggcctggggaggctggcaccgagaggagaggagtgccgagcgcagcgcatggctcccgcccgcgcccaggggccggctgtgctggggaaggaccacagctggaggagggctctgcagtgagcaaggagctggacaggcccaagagcagggagggcagagggcaggcggcgctggcagggaggggccggaggtgagctcaggaagccgggccaggaggacctgagcgggtagggggcccagaggcacatgcaggaggctgggctggggacacacagggatgggaggggagggggacaagaagactcccagggatggggtccagtacacggatggagtccagggaagggacggggtccaggacagggaagggatgggttcaggacagggatggggtcgagggcaaggacaaagcccaaggcatgaacagggttcaagacaggatcaggatcaggggagcgttggggacaagtactcctggtgctgtggctgtggagctgactcagatgacacgtggcctggcgagcagagacaggccatggcaaagtcactgagctgaggggccgggagacaacagggacagtgcggcgcagaggcccaccacacctgaagagatgcccaatcgcccagggctgggccaggctggggccacaggacagcagattagggtccatcagaagccgaccagaagcacaggtctctggtccgggctcagactggggactcagtcaggcgggccacctgcaggaggcctgagcagggacaaggggcaggggccccagggggctctaatctaggttctctgagcctgtggctgcagaggcaccagccctgatgacaggcagccacaaggggcatctaggcctcagactctctggaaacacacgggcgcaggggcaggccctggtggtcacacgcctcctctcttgcag

SEQ ID NO: 4 - DNA, 9313 bp, Sus scrofa
ggtatatatacacactagaataccactcggccgtaaaaacgaaccaaataatgccattggcagcaacacggatggaaccagagactctcatactaagtgaagtaagtcagaaagaccaataccatacgctatcacgtacatctggaatctaatatatggcacaaataaacctttccacagaaaagaaacccatggacttggagaaaatacttgtggtcgccaagggtgaggtagtggggtagactgggagtttggggttagtagatgcaacatattgcatttggcatagataaactatgagatcctgctggagagcacagggagctgtatctagtcccttgtgatggaacatgttggaggataaagtgagaaaaagaacatataaatatatacaccacacgcacacacacacacatatatgaatgactgggtcactttgccgtagagtagaaattgacagaacacagtaaatcaactataacggaaaaaaataaaaaacattaaaaaaaaaaaaacagatgctcatctccatggattctgaacacatttttgacagaattcaacacccaactttttcttgatgatttttattttttccactgcaggtggtttaccctgctctgttgattccactgtacagaaaagtgacaaagtcacacacatatatatatgtagacattctttttctcacattatcctccatcatgccccatcataggtgactagatagagggaaactacaccaacatactaaaagccatatatgacaaacccacaactatcatcattctcaatggtgaaaacctgaaagcatttccgctaagatcaggaacaagacaaggatgtctgctctcaccactctccttccacggagttttggaagtcctagccagggcaattagagaagaaaaggaatccaaatcggaaacggagaagtaacactaccgctctttacaggtgacgtgctgcgatacctagagaatcctcaaggcactaccagaaacctcttagagctcgccaatgaatttggtcaagctgcaggatacaaaattaacacacagaaattggctgccttgctctacgttaacaatgaaagagcagcgagagaaactagggaaaccatcccactgagcagtgcctcgaaatgaatgaaatgcccaggaataaacctacccaaagacacaaagacctggactctgaaaaccataaggcactgctggaagcaaccaaagacgacacaaaccggtggaaagctagaccatgctcctgccctggaagaattaatactggcaaaagggccataccacctaaggccaccctcactctcagtgcaatccctctcaaatctccaatggcattttccacagaactagaacaaaaaacctcaaaaatttgcatggaaacacaaatacctcactgacctgggctatactacaaagctgcagtcatcaaaacggtatggcactgacactcccccccaaaaaagacatacatatcagtggaacagaacagaaagcccagcaataaacccaagcacctatgctcaatccatccatgacataggaggcaagactattcaacagagaagggagagtcacttcactaagtggtgctggggagctggacagctccatgtaaagaatgaaaccagaacactcccaaacgccaaacacaccaaaaaaaaacaaaaaacaaaaaaaaaaaaataaaaaaaccctcaaaacaaatgaaactcctaaacgtaagactggatacctataactcacagaggaaaacaggcagaacgttctttgacataaatcacagcaacatcttatttgatccacctcctggaataactacaataaagacaaaagtggagttcccgtcacggctcagtggtaaccaaatctgactaggaaccatgaggttgcgggttcaatccctggccttgatcagtgggttaaggatccggcgttgccgtcagctgtggtgtaggttgcagacgcggctcggatcccacgttgctgtggctctggtgtaggccgacagctacagctccaattcgacccctagcctgggaacctccacatgccatgggtgcggccctggaaaagacaaaaagacggggaaaaaatgaaagaaagaaagaaggaaagaaggaaaggaggaaagaagaaagaaagaaagaaagaaagaaagaaagaaagaaagaaagacagacagacaaaagtaaaccaatgagatctaattaaactcaaaagcttttgaaaagcaaaagaaaccattttaaaagagaaaaacaggagttcccgtcctggctcaggggttaacgaatctgactagcatccatgaggaggcagatttgatccctggcctcactcagcaggttaaggatccggcattgccatgagctgtgagctgtggtgtaggccagcagccgtagctccaattcaacccctagcccgggaacctccatatgccacaggtggggccctaaaaaccaaaaaagacaaaggaaaacaacccacagaatgggacaaaatctttgcaaacaatgcagcccaccaaggctcaatccccaaaatatacaaacactctacaactcaacaacaacaaaaaacaaaccaacaaacaacccaatcgataaatgggcagaagacctaaatagacatttctccaaagaagacatacagatggccagaagacacatgaaaaaatcctcaacagcactaatttttagggaaatgcaaatcaaaactgcaatgaggcaccacctcacactggtcagaatggccatcattaagagtcaactaacagcaaatgctggagagggtgaggagaagagggaacccgccttcactgttggtgggaatgcaacttggtacaagcactgtggaaaacagtatggaggaacctcagaaaactgaacgtaaaaccacgattgaacccagaaatctcacccctaggcctgtatctcgatacgactttcagtcaaaaacttacatgcacaactatggactttgcagaagggttcaaaatgggcaggacatgaaaacactcaaacgcccatcaacagacgaacggattaagatgtggttctcacacaaagtaagaactacgtctcagccgtaaaaaaggaccaagtcacgccatttgcagcaacgtggatggaactagagactctcacactgagtgaagtaagtcagaaagagaaagacagagaccacatgataccacttatatgtggaatctaaaatacggcgcgattgatcctgtggacaaaacagagacagagcatggtcatggagagcagactttgctttccagggggagggagagaagagattgacagggggtttggagctggtagatgcaactgatcacattgaaaacgagtaagtgatgggtcctactgggcagcacacggaacagtgcccggcctcttgcgttagaacgtgagagaggaaggagagtgtgaaaagaaaaagaaggtaaatgtatgtacagctgtacaagggtaattgaaggaacgttgtaaatcaactctggttttaaaaaaaaaaaaaaaaatttaaagaaagaaaacgttctccagaggcagaacgagaaggtggaacggccgacaggtctctgtcctgcagcggccgctgacacgaccccaggacagcggaaatgagacccaggctgcctgtcgggcacacggcggccgccccggcaccctggtgatcctgtgcagcacgaggtgcccagtagaggggtgggcagggcagggcagggcagggcaggacctggggacgcggccgcagcaggtgggctccagccaggaaggagccacgagtggggtggggtctgctcgctgggctggaggcagggagggcacctcggctccaacatcccagccggggacccggccagaggaaggggctgcgaggatgtcttccaagcatctctttgctcttggaaccacgtggcgaagctttctgaaagcagaccagactgcagcaccacggtttctattgtgaagggatttttccagaaggagtgggaccttgcgaacggggatgtggacacagggtggcttctgatcaagcccagggccctgtgcagccaagtctcatggtcacgccgtcccggccggaccagcctgccccccacgcggacccggggaaggcaggggtcccatgaccagcacgagaacgactgactcgccttgacctcaagccacctccaggaccaggggcagacggcccggcaccacgaggaggaggcagagccccgggcagggcccacttgccacttcacctcgccagagagccgggcagatggcggcacgcgactcttggggcctgacggccccgagcccagacgctctcggggctttggccagactcgcagggctgctgagccaagcagaggctacaaagctccacgggcttccaggcaaccgctagagacacgtgggcacagctcgcagagccggtgacccacgggccagatgggaacctccctcctgtgccccctcaaggcgtctcccggaacggtggcctcccacggcgctggacggggctcggggcaggcccgaggcagcacggcgcccacaccgtccactggccgcttggaaggattcagccaacaccaccccaggcccgctgcacagccggggtgggggctgagacgtccctcaggggtccccagtcccagcctgatgctgaagttacaggacggcccaggggcgcccaggggacagggggagctgcggccctccacgaatgaggacagagggtccctgacccccacgcacggccggcaggtctcggtccacgcagagcagtgagcggctgcccgccagcccgctctcggccgggcaccaccttcacaacccctgggaactctgtccccacgtgggcagcacgggcctgggtctgggcagcaggctgggcccagccacacacgcgggggtcacaagaggtgcccggtggggtcgggcaaccaagggctcacactaggggcctggcacccgggctgcagtaggccaggcggcataatccccgtgggacatagagaaccacgacgtcctgagttcccgcaccctccccaccgctcacccaccccacacacccacatgagccgccagggcactgtctggactcccccaggccctctgggacagaaagcaaccagaagaaaagggaacttcaggaagcagccgggccacccggtttcaatcccattcttagtgtgcagggctgtgggcaggaaggtcgtgcctccagggaccagacacccacccgaggaagcagggctgcaggcacagagcacagacacaaagagccacatgcacctggggccagagcaggaggggcccggagcccggggcagcaggggagccacctcctcggcccccacactcggtgcgtggggttcacgaagaccaggggcaggggcagcggaggggggacgaggaggggagaccccacagcagggctgcagccgacccagcccagggaacacagcctgcacgcgggtcatgtcgcccgcccagcacagggaccagggagccctggcaggggcacgcccacgccaacagaggtggtgccccagaggccagaggaggtcggtggggccgccaggcctctgcggaacatgatgggctggggtcagaggcctgagggatctgctcggccaggttgctggaggcacagggctgggcaggggctgaggacgaggccaggatggacaggtgggtctgggcggaggactcagggaagagacggacacagcgcaggggacaaagaggtcacctggggttgcagggtcctgtcccggctgccggagcccgcttgagccccagggcaggtatgaaagcagtaactgtcgggtcccggggaaggagccagggccacgggcagcaggtgagccgggagcagagcaggcagggcgggccgggctcctgtgtgcccagagctggcagcacagggactgagcctaaagctccacaggccgcaggagggctgacgggagccaggggcctgagggcaggagcgggagccaggggctgggagctgggcgccagtgcagctgagccagcaactagagggagccagggaccagacaggctccaagggtggtcgcggcgagaagagggtgagctcagaagggtcaggagcccctccgggaggggaccaggtgggacagaggctctgccagggggacgctacccagagagggtgggcacccagggctccgggaacaaaggccagccctgggaaagaaaggaagccaggagtggagctagacaaggctgagggttcgggggcaggaagaggggtggtgacactgaggcccaggagccccagggaaaggggcaggtaggctgcaggtgagcagggaaccgggagggcggaaaagctgtccttagagctgctgggaacacatagagctggtgcaggtgaggaggggttgggagggcaagggggctgaacctgaagctcctcggaactggtagagcttgtgcagatgagcaggggctgggagggcagggggcagccctgggaccacctgagagctgtagatcttgggcaggtgagcacaaactggtagggcaggggaagaacctgcagttcctgggagcaggaggagcttgtgcaggtgagcaggggctggagggcagggggctaaacctggagctattgggaagaggttaggcttgtgcaggcgagcagggggtgggagggcgagggtcagtcctgggagccctttcgagccaatagagcccaggcaggtgagcaggggcttggcgggcagggagcatccctgggatgagtttgtgcaggtgagcagggggtgggagtgtagggagcagtcctagcaactcctgggagctggtagagcttgggcaggtgaacaggggctggaagtgcagggggcacatctgggaggccctgggagcaattagcgctctggcaggtgagtaggggctcggagggcagggagcagccctgggagctccttggagcaggtagagcttgggcaggtgagcagggggtgggagggcagggggcagtcctagcaactcctgggagcttgtagagcttgggcaggtgagaacgagctggtagggcaggggaatgaacctggagttactgggtcaggaggagcatgtgcaggtgagcaggggctggagggcagggggctaaacctggagctattgggaagaggttaggcttgtgcaggcaagcagggggtgggagagcaagggtcagtcctgggagcccttgcgagccggtagagcccaggcaggtgagcaggggctgggcgggcagggagcagtcctagcaactcctgggagcaggtagagcttgtgcaggtgagcagggggtgggagggtagggagcagtcctgggaactcctgcgagcaggtagagcttgtgcaggtgggcaggggtgggagggcagggggctgaacctggagctcctcagaactggtagagcttgtgcagatgagcagtggctgggagggcagggggcagccctgggagcaatagaccttgggcaggtgagaacgagctggtagggcagtggaatgaacctggagttactgggtcaagaagagcttgtgcaggcaagcaggggctgaagggcagggggctaaacctggagctattgggaagaggttaggcttgtgcaggcgagcagggggtgggagggcaagggtcagtcctgggagcccttgcgagccggtagagcccaggcaggtgagcaggggctgggagggcagggagcatccctgggacctcttgggagcaggtagagcttgtgcaggtgagcagggggtgggagggtagggagccgtcctagcaactcctgaatctggtagagcttgcggaggtgaacaggggctgggagtgcagggggcagtcctgggagcccctgggagtaattagagcttgggcaggtgagcaggagcttggagtctagggggctaaactggagctattgggaagagtttaggcttgttcaggtgagcaaggggtgggagtagagggggcattcctaggagctcccgcgagttggtcaatctcgggcaggtaagcaggggctcagaggcctggagcagccctgggaccatctgggagcagggcgagcttgggcaggtgagccgggccaggggggatgcaggacagggcaggggcaacagagtgggggtgagctcaggggagccccaggctcgagggaggggccgatatagggctagccaggctggaaagtgggctcctggggggaaggtccccaggactgcgggggcaggggcagggctgaacagacgccatgggtcacagggatcgggccaacgggccataccctgttccagcaaagtgagtggacatgggacagagcagctggagtcctaagtgagaacagcggcacagggcagtgcacatggtcggggggtcgaggggctcatctgggcctgagctggggaagcaggcaatggctgaacagaacaggggggagaggaccggcccaaccgggcaggggcgcatggggtgctggcgccccgtgggagaggaaggccaggggcagggctcggctgcagcagtgctgccaggcaggctggcagcgggaagggcaggagcagagggagggctctggcctcagcaggcagctgggaggcccagagctgggctgcaggggccggggctctggggaaagctgcctcgggtgagctcagggccaagccaggagccccggtggaggagggaccggtgggcccagaggcccagatttggagcctgggccagggccggagggatggcaggggaggggcctcggaggagactacctgccctcgcccagggaaaggggcatgttggtgccagggaccaacgcgcacgggccttgactgcagagctgacccagaggaacggggcctggagagcaaacagcaggccaggaccaggggccaggagcagagcatgggcagtgggtggcagtgggtggcagtgtccctgccaggctggggaagcaggacggtggcctcaggggaccagcagaagctggccaggaacacaggccacccgctggtcgggccaggcacccacaggggctctgggccctggcgggctctcatccaggcggtcacccagctctgaccaggcagctgcgcccagaacctgcagctgcaatggcagcgagctgggcggctggtctgccgactttctggaagcaagtgggtgctgggcgcagcggccccgctgttctgagggcccgattcgctgcccgccccacaaggaacaaggccctggcggtcacgcagcctcctctcttccag

SEQ ID NO: 5 - DNA, 337 bp, Bos taurus
tccactagggtggccaggcacagtcaccagagggggagggctcgggtgcaggggtgcgggagggtgggggaggcagcggtgtttgtgtcctcttgtttttctctttcttctcaagccccctgcacctcatcacctgctgaaacatccaaaatagctctaggtggctactgagtcattgcgagcacagcccaacccaggtgtcccagccaggctgctcttctgagaatcgggccccaaaaccgagacctggccaggtgggcctggggcctgggcccggggccaaagcccaggggagtcctacgggggcagtgagttccccaaggcctggagagggccc

SEQ ID NO: 6 - DNA, 246 bp, Bos taurus
ccccaccccctaggcaggtgcgaggccctctcagtttcccccaggttactcatttggggcacactcagccttgcagggcatgcaaatggctgtttgttccacactgaaaaacatgtctaagcctctgtggttatttccagaaatagcctacgcccacgccccacctgcagccccagctctgaccctccagagtgccaggctggcctggagctcaggattcgggggaccctgcaccccctgccccag

SEQ ID NO: 7 - protein, 145 aa, Homo sapiens
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSEKEEREREWEEEMEPTAARTRVP

SEQ ID NO: 8 - protein, 123 aa, Homo sapiens
MACRCLSFLLMGTFLSVSQTVLAQLDALLVFPGQVAQLSCTLSPQHVTIRDYGVSWYQQRAGSAPRYLLYYRSEEDHHRPADIPDRFSAAKDEAHNACVLTISPVQPEDDADYYCSVGYGFSP

SEQ ID NO: 9 - DNA, 177 bp, Homo sapiens
gtcacatctggactcccccaggccctctgagacagaaaacactcagaagaaaagggaacttcaggaagcaagtcgcgtcaccaggtttcattcctgttcttagtcttcacagcactgggggaagggccctcacgcctcctgggactggtgaccaagtcccagggagcagggctgcag

SEQ ID NO: 10 - protein, 476 aa, Bos taurus
EGESHPRVFPLVSCVSSPSDESTVALGCLARDFVPNSVSFSWKFNNSTVSSERFWTFPEVLRDGLWSASSQVVLPSSSAFQGPDDYLVCEVQHPKGGKTVGTVRVIATKAEVLSPVVSVFVPPRNSLSGDGNSKSSLICQATDFSPKQISLSWFRDGKRIVSGISEGQVETVQSSPITFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNAVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTEGEVSAEEEGFENLNTMASTFIVLFLLSLFYSTTVTLFKVK

SEQ ID NO: 11 - protein, 471 aa, Equus ferus
ESTKTPDLFPLVSCGPSLDESLVAVGCLARDFLPNVITFSWNYQNNTVVRSQDIKNFPSVLREGKYTASSQVLLPSGDVPLVCTVNHSNGNKKVEVRPQVLIQDESPNVTVFIPPRDAFTGPGQRTSRLVCQATGFSPKEISVSWLRDGKPVESGFTTEEVQPQNKESWPVTYKVTSMLTITESDWLNQKVFTCHVEHQQGVFQKNVSSMCSPNSPVPIKIFAIPPSFAGIFLTKSAKLSCQVTNLGTYDSLSISWTRQNGEILKTHTNISESHPNGTFSALGEATICVEDWESGDDYICTVTHTDLPFPLKQAISRPDAVAKHPPSVYVLPPTREQLSLRESASVTCLVKGFSPPDVFVQWLQKGQPLSSDKYVTSAPMPEPQAPGLYFVHSILTVSEEDWSSGETYTCVVGHEALPHVVTERTVDKSTEGEVSAEEEGFENLSAMASTFIVLFLLSLFYSTTVTLFKVK

SEQ ID NO: 12 - protein, 476 aa, Ovis aries
ESESHPKVFPLVSCVSSPSDENTVALGCLARDFVPNSVSFSWKFNNSTVSSERFWTFPEVLRDGLWSASSQVALHSSSTFQGTDGYLVCEVQHPKGGKTVGTVMVVAPKVEVLSPVVSVFVPPCNSLSGNGNSKSSLICQATDFSPKQISLSWFRDGKRIVSDISEGQVETVQSSPTTYRAYSVLTITEREWLSQSAYTCQVEHNKETFQKNASSSCDATPPSPIGVFTIPPSFADIFLTKSAKLSCLVTNLASYDGLNISWSHQNGKALETHTYFERHLNDTFSARGEASVCSEDWESGEEYTCTVAHLDLPFPEKSAISKPKDVAMKPPSVYVLPPTREQLSLRESASVTCLVKGFAPADVFVQWLQKGEPVAKSKYVTSSPAPEPQDPSAYFVHSILTVTEEDWSKGETYTCVVGHEALPHMVTERTVDKSTEGEVSAEEEGFENLNTMASTFIVLFLLSLFYSTTVTLFKVK

SEQ ID NO: 13 - protein, 477 aa, Sus scrofa
ESQSAPNLFPLVSCVSPPSDESLVALGCLARDFLPSSVTFSWNYKNSSKVSSQNIQDFPSVLRGGKYLASSRVLLPSVSIPQDPEAFLVCEVQHPSGTKSVSISGPVVEEQPPVLNIFVPTRESFSSTPQRTSKLICQASDFSPKQISMAWFRDGKRVVSGVSTGPVETLQSSPVTYRLHSMLTVTESEWLSQSVFTCQVEHKGLNYEKNASSLCTSNPNSPITVFAIAPSFAGIFLTKSAKLSCLVTGLVTRESLNISWTRQDGEVLKTSIVFSEIYANGTFGARGEASVCVEDWESGDRFTCTVTHTDLPSPLKQSVSKPRGIARHMPSVYVLPPAPEELSLQEWASVTCLVKGFSPADVFVQWLQKGEPVSADKYVTSAPVPEPEPKAPASYFVQSVLTVSAKDWSDGETYTCVVGHEALPHTVTERTVDKSTEGEVSAEEEGFENLNTMASTFIVLFLLSLFYSTTVTLFKVK

SEQ ID NO: 14 - protein, 471 aa, Artificial Sequence
  chimeric IgM
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVETVQSSPITFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNAVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTEGEVSAEEEGFENLNTMASTFIVLFLLSLFYSTTVTLFKVK

SEQ ID NO: 15 - protein, 614 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPT

SEQ ID NO: 16 - protein, 878 aa, Artificial Sequence
  Made in lab - EGFR-ECD-linker-hFc fusion construct
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTSRADYKDDDDKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAAEWESNGQPENNYKTTPPVLDSDGSFFLYSKLTVDKSRWQQGNVFSCSVMHEALHNHYTQKSLSLSPGK